(19) United States
(12) Patent Application Publication
(10) Pub. No.: US 2021/0138039 A1
(43) Pub. Date: May 13, 2021

(54) PAIN RESPONSE MEDIATOR COMPOSITIONS AND METHODS FOR MAKING AND USING SAME

(71) Applicant: Q-STATE BIOSCIENCES, INC., Cambridge, MA (US)

(72) Inventors: Christopher Werley, Boston, MA (US); Owen McManus, Cambridge, MA (US); Annika Malmberg, Cambridge, MA (US)

(21) Appl. No.: 16/491,917

(22) PCT Filed: Mar. 9, 2018
(86) PCT No.: PCT/US2018/021795

§ 371 Date: Sep. 6, 2019

Related U.S. Application Data
(60) Provisional application No. 62/469,010, filed on Mar. 9, 2017.

(51) Int. Cl. A61K 38/20; A61K 38/18; A61K 31/7076; A61K 38/04; A61K 38/48; A61K 33/00; A61K 35/30; A61K 38/17; A61P 29/00

(52) U.S. Cl. CPC A61K 38/204; A61K 38/185; A61K 38/18; A61K 31/7076; A61P 29/00; A61K 38/48; A61K 33/00; A61K 35/30; A61K 38/17; A61K 38/043

(57) ABSTRACT

Methods for making and using pain mediator compositions are described herein. These pain mediator compositions can be used to discover and develop pain therapeutics and to evaluate test compounds for their effect on pain associated with a particular disease or condition.

### CROSS-REFERENCE TO RELATED APPLICATION

This application claims priority to U.S. Provisional Application Ser. No. 62/469,010, filed Mar. 9, 2017, the contents of which are incorporated by reference.

### TECHNICAL FIELD

The disclosure relates to compositions comprising mediators of pain responses associated with a disease state and methods for making them. The disclosure further relates to methods for using pain mediator compositions for the discovery and development of diagnostic and therapeutic products and services.

### BACKGROUND

In the United States, the Center for Disease Control estimates that as many as 100 million people suffer from chronic pain. One prevalent approach to the treatment of pain involves the use of opioids. Although opioid drugs are highly effective treatments for pain, the abuse of those addictive drugs is understood to be an epidemic problem. There exists a major need for new therapeutics to treat and manage pain, particularly chronic pain.

One common medical condition that often induces severe chronic pain is osteoarthritis. Osteoarthritis occurs when the cartilage in joints breaks down or wears away, eventually leading to exposed bone on the joint surfaces that may rub together and can fragment or splinter. Osteoarthritis is accompanied by inflammation, which causes pain independent of the effects of bone-on-bone interaction in the joints. Many people who suffer from osteoarthritis are familiar with the enduring pain that this condition brings. Various approaches to treating the pain each comes with downsides. Over-the-counter non-steroidal anti-inflammatory drugs (NSAIDs) may be inadequate for some people. Opioids are addictive narcotics. Joint repair through, e.g., the injection of hyaluronic acid, is a costly in-patient procedure and has limited effectiveness. New approaches are urgently needed as the population in the United States and other developed countries ages, resulting in an increase in and the incidence and prevalence of osteoarthritis.

Another common medical condition that may cause severe chronic pain is cancer. The American Cancer Society attributes cancer pain to the cancer itself, not to any inflammatory or other response to the cancer. Research indicates that cancer cells themselves drive hypersensitivity of sensory neurons. Thus, not only can a cancer manifest as a tumor, or spread throughout the body, but the cancer itself can be a direct cause of severe pain. Currently, the standard treatment for cancer pain is use of opioids. Not only are opioids addictive, they have the additional disadvantages that they lose effectiveness over time and cause respiratory depression and constipation. New therapeutic approaches are needed to treat and manage cancer-induced pain.

Similarly, neuropathic pain is difficult to treat and often does not respond to conventional pain medications or other analgesic therapies. As opposed to nociceptive pain, which is felt as the result of an acute injury, neuropathic pain does not resolve quickly and can be accompanied by a loss of sensation or numbness. Neuropathic pain can be associated with an injury or disease that affects parts of the sensory system, such as peripheral nerve tissue. In addition to trauma to nerve tissue, certain conditions can predispose a subject to developing neuropathic pain, including diabetes, cancer, multiple sclerosis, and viral diseases such as HIV. Due to the lack of effectiveness of standard analgesics or other approved therapies, other medications are sometimes used off-label to treat neuropathic pain including certain antidepressant drugs (such as certain tricyclic antidepressants and selective serotonin reuptake inhibitors) and anti-seizure medications (such as gabapentin). There remains a well-recognized need for new therapies to treat neuropathic pain.

### SUMMARY

The disclosure provides a method of making a composition comprised of molecules that are selected to mediate a pain response in excitable neurons in a manner that reproduces the microenvironment present in a target tissue associated with a pain response, for example, a tissue that initiates a pain signal to the brain. Such pain signals may be different in specific diseases or conditions, e.g., osteoarthritis, cancer, or neuropathy. Thus, the compositions of the disclosure may induce neurons to respond to different types of pain, e.g., inflammatory pain, cancer pain, or neuropathic pain. When neurons are contacted with such compositions, whether situated in a cell culture, excised tissue (e.g., slice), organoids, animal models, etc., the neurons exhibit features associated with the type of pain under study. Thus, the compositions can be used for discovery, research and development of new analgesic therapies or to evaluate the potential efficacy of products to treat a particular disease state or condition.

The selection of ingredients for a pain mediator composition of the invention may include choosing molecules for which one or more of the following is true: (i) the molecule triggers an action potential in a neuron; (ii) the molecule increases sensitivity to a pain response in a neuron; (iii) the molecule triggers a pain response in an animal (e.g., irritates the skin); (iv) when the molecule is administered to an animal for which the associated receptor gene is knocked out, there is no pain response; and (v) the molecule has been found in a target tissue of interest (including without limitation, diseased or injured tissue, inflammatory sites or tumors). An important insight of the disclosure is that molecules involved in a pain response are unlikely to operate in isolation; instead, each molecule likely participates in a complex feedback loop and/or signal cascade. Therefore, selection of the types and concentrations of molecules associated with a pain response may help to formulate a composition of the invention that can model pain responses of neurons associated with a particular disease or condition, and can therefore be used to test for agents that may alleviate pain associated with the particular disease or condition. The composition may advantageously comprise pain mediators that are combined in relative concentrations that approximate the relative concentrations of such mediators in a particular target tissue, such as a tumor, damaged or diseased nerve tissue or tissue affected by inflammation (e.g., osteoarthritic tissue).

The disclosure further provides a method to model a pain response using neurons exposed to media that includes a plurality of mediators that are selected to mimic the local environment of a tissue associated with a specific pain signal, such as an osteoarthritic joint; a tumor; or other disease or condition that causes pain. The disclosed pain models may be used to discover or develop therapeutic agents for pain associated with a particular disease or condition, or to predict the analgesic effects of test compounds.

The in vitro pain models of the invention can be performed in different ways, including in wells, on slides, or in other fluid partitions, or can be performed in a high-throughput assay to screen through a large library of compounds to identify compounds which may have potential as analgesics. In these models, compositions of selected pain mediators are introduced to cultured neurons, which then exhibit greatly increased rapidity of firing and hypersensitization. Sensory neurons such as dorsal root ganglion neurons are known to send pain signals to the brain. If one exposes sensory neurons such as dorsal root ganglion neurons to the disclosed pain mediator compositions, then one can model neuronal signals that would be experienced by the brain as pain. One may then test compounds in the model to identify novel compounds that return neuronal signals to a baseline state in the presence of the pain mediator composition. Compounds active in these assays would have potential as analgesics. Initial results have identified compounds that counteract the effects of an inflammatory pain mediator composition and return neural firing to a baseline state. Those results confirm that novel analgesics to different types of pain may be found by using the disclosed pain models, which drugs may be effective for the treatment of chronic pain that is associated with specific diseases or conditions, such as cancer, neuropathy or inflammatory conditions such as osteoarthritis.

In certain aspects, the invention provides a method that includes (a) selecting pain mediators based on their presence in a target tissue associated with a pain response; and (b) combining the mediators to create a pain mediator composition. The pain mediators may be combined in relative concentrations that approximate the relative concentrations of such mediators in the target tissue. The target tissue may be, for example, a tumor. The mediators may include four or more of nerve growth factor (NGF), endothelin 1 (ET-1), tumor necrosis factor alpha, interleukin 6, adenine triphosphate, bradykinin, acidic pH, and a protease. In some embodiments, the target tissue is nerve tissue. The mediators may include four or more of tumor necrosis factor alpha, nerve growth factor (NGF), interleukin (IL)-1beta, IL-6, IL-17, acidic pH, PGE2, and CCL2. In certain embodiments, the target tissue is affected by inflammation and may be, for example, osteoarthritic tissue. The mediators may include four or more of tumor necrosis factor alpha, nerve growth factor (NGF), interleukin (IL)-1beta, IL-6, IL-17, acidic pH, PGE2, and CCL2. In particular embodiments, specific mediators identified in the compositions and methods disclosed herein are optionally exchanged for other mediators that behave similarly (i.e., are classified in the same category).

In some embodiments, the mediators include at least one interleukin, at least one growth factor and at least one other mediator. The method may include (c) establishing a baseline neural activity for neurons not exposed to the pain mediator composition; and (d) measuring a neural response from neurons exposed to the pain mediator composition to thereby demonstrate that the pain mediator composition mediates a pain response. Establishing the baseline and measuring the neural response may be performed in vivo using an animal model. Optionally, establishing the baseline and measuring the neural response include detecting an optical signal from the neurons in vitro.

Selecting the mediators may include identifying mediators that satisfy one or more of the following: (a) the mediator triggers an action potential in a neuron; (b) the mediator increases sensitivity to a pain response in a neuron; (c) the mediator triggers a pain response in an animal model; (d) when the mediator is administered to an animal for which the associated receptor gene is knocked out, there is no pain response.

Aspects of the disclosure provide a composition that includes a plurality of pain mediators that are present in a target tissue associated with a pain response. Preferably, the pain mediators are present in relative concentrations that approximate the relative concentrations of such mediators in the target tissue. In some embodiments, the target tissue is a tumor. The mediators may include four or more of nerve growth factor (NGF), endothelin 1 (ET-1), tumor necrosis factor alpha, interleukin 6, adenine triphosphate, bradykinin, acidic pH, and a protease.

The target tissue may include nerve tissue. The mediators may include four or more of tumor necrosis factor alpha, nerve growth factor (NGF), interleukin (IL)-1beta, IL-6, IL-17, acidic pH, PGE2, and CCL2.

In certain embodiments, the target tissue is affected by inflammation or is osteoarthritic tissue. The mediators may include four or more of tumor necrosis factor alpha, nerve growth factor (NGF), interleukin (IL)-1beta, IL-6, IL-17, acidic pH, PGE2, and CCL2.

Preferably, the mediators include at least one interleukin, at least one growth factor and at least one other mediator. In some embodiments, the mediators satisfy one or more of the following: (a) the mediator triggers an action potential in a neuron; (b) the mediator increases sensitivity to a pain response in a neuron; (c) the mediator triggers a pain response in an animal model; and (d) when the mediator is administered to an animal for which the associated receptor gene is knocked out, there is no pain response.

In certain embodiments, administration of the composition to an animal causes a pain response in the animal.

The composition may further include living neurons. The neurons and the plurality of pain mediators may be provided in media ex vivo. Optionally, at least one of the neurons includes an optical reporter of neural activity (such as a microbial rhodopsin protein).

In certain aspects, the disclosure provides a method that includes inducing a pain response in a neuron by contacting the neuron with a pain mediator composition which includes pain mediators that are present in a target tissue associated with a pain response. The pain mediator composition may include a plurality of soluble proteins. The method may be performed in vivo by administering the pain mediator composition to an animal. For example, the pain response includes pain-associated behavior by the animal.

In certain embodiments, the method includes contacting the pain mediator composition with a neural sample in vitro, to neuronal slices, to a tissue sample, or to organoids. The pain response may include increased firing by the neuron. The neuron may include an optical reporter of neural activity and the method may include optically detecting the increased firing by the neuron.

In certain embodiments, the pain mediator composition includes a plurality of mediators selected based on their presence in or near a tumor. For example, the plurality of mediators may include a plurality of: nerve growth factor (NGF), endothelin 1 (ET-1), tumor necrosis factor alpha, interleukin 6, adenine triphosphate, bradykinin, acidic pH, and a protease.

In some embodiments, the pain mediator composition includes a plurality of mediators selected based on their presence in or near inflamed tissue. For example, the mediators may include a plurality of tumor necrosis factor alpha, nerve growth factor (NGF), interleukin (IL) 1beta, IL-6, IL-17, acidic pH, PGE2, and CCL2. Optionally, the pain mediator composition includes a plurality of mediators selected based on their presence in or near nerve tissue. The mediators may include a plurality of tumor necrosis factor alpha, nerve growth factor (NGF), interleukin (IL)-1beta, IL-6, IL-17, acidic pH, PGE2, and CCL2.

Aspects of the disclosure provide a method for developing or discovering a therapeutic for pain associated with a disease or condition. The method includes contacting a pain mediator composition developed for the disease or condition with a neuron and contacting a therapeutic to the neuron to determine an effect of the therapeutic. Contacting the pain mediator composition with the neuron may further include recording a pain response of the neuron to the pain mediator composition. The method may include measuring the ability of the therapeutic to ameliorate the pain response. The steps may be performed in vivo on in an animal model. The pain response may be measured by observing animal behavior.

In some embodiments, the steps are performed in vitro on a sample that includes neurons. Contacting a pain mediator composition with a neuron may further includes recording pain response of the neuron to the pain mediator composition. The neurons may include an optical reporter of neural activity (e.g., a microbial rhodopsin protein), an optical actuator of neural activity (e.g., an algal channelrhodopsin), or both.

The method may include determining the effect of a therapeutic to a disease or condition by recording an action potential waveform using the optical reporter of neural activity. Preferably, the pain mediator composition includes a plurality of soluble proteins. The disease may be cancer. The mediators may include four or more of nerve growth factor (NGF), endothelin 1 (ET-1), tumor necrosis factor alpha, interleukin 6, adenine triphosphate, bradykinin, acidic pH, and a protease.

The condition may be neuropathy. The mediators may include four or more of tumor necrosis factor alpha, nerve growth factor (NGF), interleukin (IL)-1beta, IL-6, IL-17, acidic pH, PGE2, and CCL2.

In certain embodiments, the condition is inflammation such as may be associated with osteoarthritis. The mediators may include four or more of tumor necrosis factor alpha, nerve growth factor (NGF), interleukin (IL)-1beta, IL-6, IL-17, acidic pH, PGE2, and CCL2. Preferably, the pain mediators include at least one interleukin, at least one growth factor and at least one other pain mediator.

Aspects of the disclosure provide a method of evaluating a test compound for effect on pain associated with a disease or condition. The method includes providing a sample comprising one or more neurons in media comprising a plurality of pain mediators associated with the disease or condition and measuring an effect of the test compound in the presence of the pain mediators on neural activity in the sample. The pain mediators may include molecules that have been shown to have increased expression in or near tumors or an inflammatory condition in humans, relative to healthy blood or tissue. The molecules may include at least one interleukin, at least one growth factor, and at least one other mediator. The method may include establishing a baseline neural activity for neurons not exposed to the pain mediators; measuring a neural response to exposure to the pain mediators to determine an effect of the pain mediators; comparing the effect of the pain mediators to the effect of the test compound; and identifying the test compounds as potential analgesics when the effect of the test compound in the presence of the pain mediators is restorative to the baseline. Preferably, during the measuring, at least one of the neurons lives in the media within a measuring instrument that includes a control unit operable to control at least temperature or pressure above the liquid media. The measuring instrument may include an optical microscopy system operable to optically trigger an action potential in at least a first neuron in the media, to optically excite a fluorescent reporter of action potential in the neurons, and to optically detect an action potential reported by the reporter. The neurons may include actuator that initiates an action potential in response to incoming light and/or at least one reporter that emits light in response to an action potential. Measuring neural activity may include measuring an optical signal emitted by a neuron of the one or more neurons. In certain embodiments, the one or more of the one or more neurons include optical actuators of neural activity, optical reporters of neural activity, or both. Measuring neural activity may include recording an action potential and storing a representation of an action potential waveform.

The method may include comparing the effect of the test compound on neural activity in the presence of the pain mediators to an effect of the pain mediators on neural activity in the absence of the test compound by comparing action potentials of neurons under the respective conditions. Comparing the action potentials may include determining differences between one or more of spike width, after-hyperpolarization depth, stimulation intensity at maximum spike rate, maximum spike rate, and number of spikes under a fixed period. The method may include obtaining a baseline firing phenotype for neurons not exposed to a plurality of pain mediators.

In some embodiments, the method includes measuring a response of neurons in the presence of the pain mediators to a tool compound having a known analgesic effect in humans; and identifying the test compound as a potential analgesic when the measured effect of the test compound in the presence of the pain mediators correlates with the measured response of the neurons to the tool compound. The measuring may be repeated for a plurality of test compounds to identify at least one active compound for which the effect measured in vitro predicts an in vivo analgesic effect.

The pain mediators may include at least one nociceptive trigger and at least one modulator of sensory processing. In certain embodiments, the plurality of pain mediators include three or more mediators, each independently selected from the group consisting of an interleukin, a growth factor, a protease, a tumor necrosis factor, and protons. The plurality of pain mediators may further include a plurality of further mediators selected from the group consisting of histamine, serotonin, ATP, bradykinin, prostaglandin E2, and endothelin-1.

In some embodiments, the plurality of pain mediators include proteins found to have increased expression in synovial fluid of osteoarthritis subjects relative to blood or serum of a healthy subject. The plurality of pain mediators may include proteins selected from tumor necrosis factor (TNF), IL, PGE2, substance P and a growth factor; a signaling mediator selected from NFkB, ERK1/2, p38, JK, PKCdelta, TLRs, Beta-catenin, Gli1, Ptch, HHIP, HIF-2alpha, iNOS, and RUNX2; and a protease.

In certain embodiments, the plurality of pain mediators includes proteins found to have increased expression in a tumor microenvironment relative to blood or serum of a tumor-free subject. The plurality of pain mediators may include at least five of tumor necrosis factor alpha (TNF-a); IL-1b; IL-6; IL-1; IL-15; IL-16; prostaglandin; fragments of extra-cellular matrix; trypsin; ATP; H+; neurturin; GDNF; and BDNF.

In certain aspects, the invention provides a method of testing a drug. The method includes providing a sample comprising one or more neurons in media comprising a pain mediator composition of the invention and measuring an effect of a test compound in the presence of the pain mediator composition on neural activity in the sample. The method includes establishing a baseline neural activity for neurons not exposed to the pain mediator composition, measuring a neural response to exposure to the pain mediator composition to determine an effect of the pain mediator composition on the neurons, comparing the effect of the pain mediator composition in the presence and absence of a test compound, and identifying test compounds as active (i.e., potential analgesics) when the effect of the test compound in the presence of the pain mediator composition effects a change in the neurons towards the baseline neural activity.

In some embodiments, during the measuring, at least one of the neurons lives in the media within a measuring instrument that includes a control unit operable to control at least temperature or pressure above the liquid media. The measuring instrument may be, for example, an optical microscopy system operable to optically trigger an action potential in at least a first neuron in the media, to optically excite a fluorescent reporter of action potential in the neurons, and optically detect an action potential reported by the reporter. In optogenetic embodiments, the neurons may include an actuator that initiates an action potential in response to incoming light and at least one reporter that emits light in response to an action potential. Measuring neural activity may include measuring an optical signal emitted by a neuron of the one or more neurons. One or more of the neurons include optical actuators of neural activity (e.g., an algal channelrhodopsin), optical reporters of neural activity (e.g., a microbial rhodopsin), or both. Measuring neural activity may be done by recording an action potential and storing a representation of an action potential waveform.

Methods may include comparing the effect of the test compound on neural activity in the presence of a pain mediator composition to an effect of the pain mediator composition on neural activity without the test compound by comparing action potentials of neurons under the respective conditions. For example, comparing the action potentials may include determining differences between one or more of spike width, after-hyperpolarization depth, stimulation intensity at maximum spike rate, maximum spike rate, and number of spikes under a fixed period. Optionally, the method includes obtaining a baseline firing phenotype for neurons not exposed to a pain mediator composition of the invention.

In certain embodiments, the method includes measuring a response of neurons in the presence of a pain mediator composition to a tool compound having a known analgesic effect in humans and identifying the test compound as an active compound (i.e., potential analgesic) when the measured effect of the test compound in the presence of the pain mediator composition correlates with the measured response of the neurons to the tool compound. The measuring may be repeated for a plurality of test compounds to identify at least one active compound for which the effect measured in vitro predicts an in vivo analgesic effect.

The pain mediator compositions of the invention may include at least one nociceptive trigger and at least one modulator of sensory processing. In preferred embodiments, the plurality of pain mediators in a pain mediator composition include three or more mediators, each independently selected from an interleukin, a growth factor, a protease, a tumor necrosis factor, and protons. The pain mediators in a pain mediator composition of the invention may further include a plurality of further molecules selected from the group consisting of histamine, serotonin, ATP, bradykinin, prostaglandin E2, and endothelin-1. It may be preferable that a pain mediator composition intended to model osteoarthritic inflammatory pain includes proteins found to have increased expression in synovial fluid of osteoarthritis subjects relative to blood or serum of a healthy subject; or that pain mediator compositions intended to model tumor pain include proteins found to have increased expression in a tumor environment relative to blood or serum of a tumor-free subject; or that a pain mediator composition intended to model another disease condition include proteins found to have increased expression in or near disease tissue inducing a pain signal relative to analogous tissue that is not afflicted with the disease condition.

In certain embodiments, the plurality of pain mediators selected for inclusion in a pain mediator composition includes a molecule selected from the group consisting of TNF-a, IL, PGE2, substance P and a growth factor; a signaling mediator selected from NFkB, ERK1/2, p38, JK, PKCdelta, TLRs, Beta-catenin, Gli1, Ptch, HHIP, HIF-2alpha, iNOS, and RUNX2; and a protease. The pain mediator composition may specifically include at least five of TNF-a, IL-1b; IL-6; IL-1; IL-15; IL-16; prostaglandin; fragments of extra-cellular matrix; trypsin; ATP; H+; neurturin; GDNF; and BDNF.

Aspects of the invention provide a pain mediator composition to model cancer pain, which includes a plurality of molecules present in or near tumor tissue at an increased level relative to healthy tissue, wherein each of the plurality of molecules is associated with a receptor expressed in a neuron, and wherein each of the plurality of molecules induces pain when injected in an animal model. Preferably, a pharmacological blocker of the receptor(s) associated with at least some of the molecules alleviates pain in an animal model. The pain mediator composition preferably includes at least two molecules selected from nerve growth factor (NGF), interleukin-6 (IL-6), endothelin-1 (ET-1), tumor necrosis factor alpha (TNF-a), bradykinin (BK), adenosine triphosphate (ATP), H+, and Trypsin. The pain mediator composition may further include to two or more of NGF at about 60 μg/mL, IL-6 at about 4 ng/mL, ET-1 at about 30 nM to about 10 μM, TNF-a at about 0.4 ng/mL, BK at about 5 μM, ATP at about 100 μM, H+ to a pH of about 6.5, and trypsin at about 1 μg/mL. The pain mediator composition may also contain at least one of formaldehyde, Substance P (SP), and brain-derived neurotrophic factor (BDNF).

In related aspects, the invention provides a method for screening a compound for pain treatment. The method includes contacting a compound with a sample comprising a neuron (e.g., a DRG neuron) in the presence of a plurality of molecules present in or near tumor tissue, an osteoarthritis lesion, or other inflamed or diseased tissue at an increased level relative to healthy tissue, measuring excitability changes in the neuron, and identifying the compound as a candidate for pain treatment based on the measured excitability changes. In some embodiments, the neuron expresses an optical reporter of membrane electrical potential (e.g., a microbial rhodopsin) and/or a light-gated ion channel (e.g., an algal channelrhodopsin) such that measuring the excitability may include receiving, via a microscopy system, an optical signal generated by the optical reporter in response to optical stimulation of the sample following presentation of said compound.

Aspects of the invention provide a method for preparing a pain mediator composition. The method includes: identifying a plurality of molecules present in or near tumor tissue, an osteoarthritis lesion, or other inflamed or diseased tissue at an increased level relative to healthy tissue (e.g., in which each of the plurality of molecules is associated with a receptor expressed in a neuron and each of the plurality of molecules induces pain when injected in an animal model); and preparing a pain mediator composition comprising the plurality of molecules. In some embodiments, the plurality of molecules includes two or more of nerve growth factor (NGF), interleukin-6 (IL-6), endothelin-1 (ET-1), tumor necrosis factor alpha(TNF-a), bradykinin (BK), adenosine triphosphate (ATP), H+, and trypsin. The plurality of molecules may further include two or more of NGF at about 60 μg/mL, IL-6 at about 4 ng/mL, ET-1 at about 30 nM to about 10 μM, TNF-a at about 0.4 ng/mL, BK at about 5 μM, ATP at about 100 μM, H+ to a pH of about 6.5, and trypsin at about 1 μg/mL. The plurality of molecules may further include one or more of formaldehyde, Substance P (SP), and brain-derived neurotrophic factor (BDNF).

The disclosure provides methods to select a plurality of molecules to include in a pain mediator composition that approximates the pain-inducing mechanisms of cancer microenvironments based on certain criteria. Such criteria may include increased levels of a molecule in or near tumors, expression of neuronal receptors associated with one or more of the plurality of molecules, and induction of local pain with injection of each molecule or plurality of molecules in animal models. Systems and methods for screening compounds are provided including characterizing the effect of compounds in cancer pain treatment by exposing neurons in a cancer pain mediator composition to a test compound.

Models of the disclosure may include in vitro cellular models for screening compounds for effectiveness in treating pain, e.g., cancer pain, inflammatory pain, or neuropathic pain. The models may include dorsal root ganglion (DRG) neurons or sensory neurons exposed to a pain mediator composition of the disclosure. In certain embodiments, the neurons may express optogenetic proteins that allow emit light in response to neural activity such that the effects of potential therapies on neurons contacted with the pain mediator composition may be optically measured.

In order to develop an accurate cancer pain mediator composition, methods of the disclosure include characterizing the cancer microenvironment by, for example, identifying molecules present at increased levels in or near various tumors in humans. Molecules found in or near tumors in multiple cancer types and locations may be considered more important components in putative cancer pain models than molecules only found at higher levels in or near individual cancer types. In order to function in in vitro models of the disclosure, molecules selected for a cancer pain mediator composition should have associated receptors on the neurons of the in vitro model. Molecules selected for inclusion in a cancer pain mediator composition should also be shown to individually cause local pain in animal models. Pain mediator compositions for pain associated with cancer chemotherapy-induced pain may be developed similarly.

In certain embodiments, molecules selected for inclusion in a cancer pain mediator composition may be determined by additional criteria. The additional criteria may include a finding that pharmacological blockers of receptors associated with a specific molecule alleviate pain when that molecule is present in animal models. Other criteria may include a finding that a knockout organism, genetically modified to not express receptors associated with a specific pain mediator, does not experience pain when exposed to that molecule.

In certain aspects, the cancer pain mediator composition may include two or more pain mediators selected from nerve growth factor (NGF), interleukin-6 (IL-6), endothelin-1 (ET-1), tumor necrosis factor alpha (TNF-a), bradykinin (BK), adenosine triphosphate (ATP), H+, and trypsin. Additional pain mediators may include formaldehyde, Substance P (SP), and brain-derived neurotrophic factor (BDNF).

Selection of the concentrations of pain mediators within a pain mediator composition may be determined by measuring concentrations within the microenvironment present in cancer, inflamed tissue, damaged nerve tissue or other tissue afflicted with a disease condition that induces pain, or may be determined experimentally through in vitro application of various concentrations and combinations of pain mediators to neurons. Neuronal responses to different combinations and concentrations of pain mediators may be monitored by, for example, detection of neural activity through illumination of optogenetic proteins. The optimal combination and concentration of pain mediators in a pain mediator composition may be determined by the amount and type of neural activity measured in response to the various combinations and concentrations of pain mediators tested. The amount and type of neural activity measured would reflect the in vivo neural activity observed in response to pain resulting from a disease which the pain mediator composition is intended to model.

Aspects provide a method of evaluating a test compound for effect on pain associated with chemotherapy. The method includes providing a sample comprising one or more neurons such as dorsal root ganglia exposed to a plurality of pain mediators associated with chemotheray and measuring an effect of the test compound in the presence of the pain mediators on neural activity in the sample. The method may include measuring baseline DRG behavior, adding a chemotherapeutic and measuring change in behavior, and screening for therapeutic candidates that reverse the behavioral change that results from the chemotherapeutics. In some embodiments, the chemotherapeutics include compounds that cause sensory neuropathy (e.g., paclitaxel), compounds that cause neuronal hyperexcitability (e.g., cisplatin), others, or combinations thereof. Methods may include synaptic assays for, e.g., hyperexcitability at different concentrations (clinical dose (CD), CD/5, & CD*5) and at time points.

Chemotherapeutic pain mediator compositions may be validated in cultured human stem cell-derived neurons with sensory neuron-like properties.

### BRIEF DESCRIPTION OF THE DRAWINGS

FIG. 1 diagrams a method of testing a drug.

FIG. 2 shows a sample with neurons and mediators of inflammation.

FIG. 3 shows a plurality of pain mediators as may be used to approximate the inflammatory microenvironment of a joint affected by osteoarthritis.

FIG. 4 illustrates a plurality of mediators of cancer pain as may be used to model a tumor microenvironment.

FIG. 5 is a taxonomy of pain mediators.

FIG. 6 shows a microscope for measuring electrical properties of cells.

FIG. 7 illustrates an illumination subsystem of the microscope.

FIG. 8 illustrates an optical actuator and an optical reporter of neural activity.

FIG. 9 illustrates the recording of action potential waveforms from neurons.

FIG. 10 shows a series of action potentials.

FIG. 11 shows a radar plot of action potentials.

FIG. 12 shows a plating strategy for screening.

FIG. 13 gives results from another instrument run, but with a test compound being tested.

FIG. 14 shows neural activity after treatment with an inflammatory pain mediator composition.

FIG. 15 shows the treatment of the neurons with a selective blocker of Nav1.8.

FIG. 16 shows treatment with a selective blocker of Nav1.7.

FIG. 17 gives a concentration response curve (CRC) for duloxetine.

FIG. 18 gives a CRC for flupirtine.

FIG. 19 gives a CRC for PF-05089771.

FIG. 20 gives a CRC for ProTx-II.

FIG. 21 diagrams an optogenetic method of testing a drug.

FIG. 22 shows certain pain mediators and concentrations in various samples.

FIG. 23 shows a plating for heterologous screening.

FIG. 24 shows an exemplary method for preparing a cancer pain mediator composition.

FIG. 25 gives a recipe for an osteoarthritis pain mediator composition.

FIG. 26 shows radar plots showing the drug-induced change in neuronal behavior.

FIG. 27 shows measured action potentials.

FIG. 28 shows neural activity.

FIG. 29 shows, for each well, the drug score.

FIG. 30 shows high signal-to-noise ratio fluorescent voltage recordings of neurons.

FIG. 31 is a raster plot showing spikes from columns of wells.

FIG. 32 shows the average firing rate during the ramp for each well.

FIG. 33 gives a heat map showing the number of spikes during the ramp for each well.

FIG. 34 shows the average number of spikes per cell.

FIG. 35 shows measurements from rat dorsal root ganglion (DRG) neurons.

FIG. 36 is a raster plot showing the resulting spike data for rat DRG neurons.

FIG. 37 shows measurements from human iPSC-derived NGN2 neurons.

FIG. 38 gives the single values resulting from rat DRG neurons and NGN2 neurons.

FIG. 39 shows an effect of a pain mediator composition of neural activity.

FIG. 40 shows synaptic assays for neural activity.

FIG. 41 gives a formulation of a cancer pain mediator composition.

FIG. 42 shows a cancer pain mediator composition phenotype.

FIG. 43 shows stem cell-derived sensory neurons.

FIG. 44 gives probe compounds expected to modulate pain signaling, excitability, or synaptic transmission.

FIG. 45 shows results from a pain mediator composition being introduced to neurons.

### DETAILED DESCRIPTION

FIG. 1 diagrams a method 101 of testing a drug. The method 101 includes providing a sample 201 that includes one or more neurons in media that includes 301 a plurality of pain mediators. An effect 509 of a test compound on neural activity in the presence of the pain mediator composition is measured 509. The plurality of pain mediators may be found at a site of inflammation in human tissue. The disclosure includes the insight that tumors, inflammatory conditions such as osteoarthritis, and other diseases that initiate a pain signal exhibit an environment that causes a sensation of pain for the person affected by the condition. Thus, these conditions give rise to their own innate “pain soup” or infiltrate in vivo. The composition of such “pain soup” may vary from case to case but some consistent patterns, such as the inclusion of interleukins, proteases, or acidity, may be found to emerge.

Those ingredients may not cause a pain sensation through a strict one-to-one, linear, causative relationship but may, instead, each independently contribute to or participate in a complex cytochemical pattern of receptor agonism and antagonism, signal cascades subject to both positive and negative feedbacks, consequential physical trauma to tissue in the form of, e.g., swelling, drying, tearing, or ablation, and other molecular phenomena not yet appreciated. As such, it may be found that little is to be gained by singling out any one ingredient of a pain causing infiltrate to identify a drug that blocks a direct effect of that ingredient. Negating the presence of one molecular species may do little to ease the very real sensation of pain through which the person is suffering.

Methods of the disclosure include measuring, in vitro, how neurons transmit signals in response to a complex mixture of mediators of pain, or “pain soup”, as a tool for understanding pain and researching potential therapies for pain arising from cancer, osteoarthritis, neuropathy, and other diseases. For the discovery of effective pain therapies, the disclosure provides an ex vivo sample that includes one or more neurons in media that includes a plurality of pain mediators that have been selected to reproduce the effects of pain mediators in vivo. The selected plurality of pain mediators comprises a pain mediator composition that may be used to model the pain associated with the relevant disease in in vitro, ex vivo, and in vivo models.

Methods and compositions of the disclosure may be used in screening for a pain therapeutic. A screen for pain therapeutics based on a pain mediator composition according to the disclosure may involve measuring baseline neuronal morphology or functional behavior (e.g., measuring electrophysiology by, for example, measuring action potentials), applying the pain mediator composition and measuring the change in neuronal morphology or functional behavior, and screening for potential therapeutics that revert neuronal morphology or functional behavior to baseline levels. The screening assay could probe a variety of morphological or functional metrics. A functional screen could probe, for example, neurite outgrowth, number of synapses, neuron size, neuron shape, or characteristics of organelle size and structure. A functional screen could probe spontaneous activity via calcium or electrical measurements. Fluorescent assays for calcium or voltage could be based on protein-based sensors or applied small-molecule dyes. Electrical measurements could also be implemented with electrodes such a multi-electrode arrays or patch clamp electrodes. Metrics for action potential shape or firing patterns may be used as an assay readout. Additionally or alternatively, integrated activity may be measured with activity-dependent promoters such as c-Fos (a proto-oncogene expressed in some neurons after depolarization). In addition to spontaneous activity, neuronal activity may be invoked electrically using optogenetic constructs or with chemical agonists. Induced activity in response to a weaker stimulus would generally be associated with hypersensitivity to pain. In addition to direct measures of neuronal excitability, measurements may be made probing synaptic activity from the sensory neurons into the central nervous system: strengthened synapses would canonically result in stronger pain signals transmitted to the brain. See U.S. Patent Application publication 2018/0031553 and U.S. Pat. No. 9,207,237.

FIG. 2 shows sample 201 that includes one or more neurons 113 in media 138 that includes a pain mediator composition 115. The sample 201 may be provided in any suitable container 111 such as a well of a multi-well plate or in a micro centrifuge tube or on a glass slide or dish. In the depicted embodiment, the sample 201 is in a container 111 with a transparent bottom 112 so that, for example, the neurons 113 may be observed from above or below.

The neurons 113 may be obtained from any suitable source. For example, in some embodiments, the neurons are differentiated from stem cells. Pluripotent stem cells may be produced by direct differentiation on a solid surface in the presence of one or more added TGF-ß superfamily antagonists, such as noggin and follistatin. Alternatively, pluripotent stem cells can be cultured as clusters or embryoid bodies. Enrichment for neural cells of varying degrees of maturity comprises culturing in a medium containing added mitogens or growth factors (such as EGF and FGF), concurrently or followed by added neurotrophin (such as NT-3 or BDNF) and other factors (such as EPO) in various optimized combinations. Some embodiments use induced pluripotent stem cells (iPSCs). iPSCs are characterized by their ability to proliferate indefinitely in culture while preserving their developmental potential to differentiate into derivatives of all three embryonic germ layers. In certain embodiments, fibroblasts are converted to iPSC by methods such as those discussed in Takahashi and Yamanaka, 2006, Induction of pluripotent stem cells from mouse embryonic and adult fibroblast cultures by defined factors Cell 126:663-676.; and Takahashi, et al., 2007, Induction of pluripotent stem cells from adult human fibroblasts by defined factors, Cell 131:861-872. In other embodiments, peripheral blood mononuclear cells (PBMCs) are converted to iPSCs. There are multiple methods to generate iPSCs from PBMCs including, for example, using viral-mediated gene transduction and chemical induction. One approach is to use reprogramming vectors such as those sold under the trademark CYTOTUNE by ThermoFisher Scientific (Waltham, Mass.). Such reprogramming vectors do not integrate into the host genome or alter the genetic information of the host cell. See Fusaki, 2009, Efficient induction of transgene-free human pluripotent stem cells using a vector based on Sendai virus, an RNA virus that does not integrate into the host genome, Proc Jpn Acad Ser B Phys Biol Sci 85(8):348-362 and Seki, 2010, Generation of induced pluripotent stem cells from human terminally differentiated circulating T cells, Cell Stem Cell, 7(1):11-14, both incorporated by reference.

Induction of pluripotent stem cells from adult fibroblasts and PBMCs can be done by methods that include introducing four factors, Oct3/4, Sox2, c-Myc, and Klf4, under ES cell culture conditions. Human dermal fibroblasts (HDF) or PBMCs are obtained. A retrovirus containing human Oct3/4, Sox2, Klf4, and c-Myc may be used for transduction. Six days after transduction, the cells may be harvested by trypsinization and plated onto mitomycin C-treated SNL feeder cells. See, e.g., McMahon and Bradley, 1990, Cell 62:1073-1085, incorporated by reference. About one day later, the medium (DMEM containing 10% FBS) is replaced with a primate ES cell culture medium supplemented with 4 ng/mL basic fibroblast growth factor (bFGF). See Takahashi, et al., 2007, Cell 131:861. Later, hES cell-like colonies are picked and mechanically disaggregated into small clumps without enzymatic digestion. Each cell should exhibit morphology similar to that of human ES cells, characterized by large nuclei and scant cytoplasm. The cells become iPS cells, which can then be differentiated into specific neuronal subtypes. See Dimos et al., 2008, Induced pluripotent stem cells generated from patients with ALS can be differentiated into neurons, Science 321(5893):1218-21; Amoroso et al., 2013, Accelerated high-yield generation of limb-innervating neurons from human stem cells, J Neurosci 33(2):574-86; and Boulting et al., 2011, A functionally characterized test set of human induced pluripotent stem cells, Nat Biotech 29(3):279-286; Davis-Dusenbery et al., 2014, How to make spinal neurons, Development 141(3):491-501; Sandoe and Eggan, 2013, Opportunities and challenges of pluripotent stem cell neurodegenerative disease models, Nat Neuroscience 16(7):780-9; and Han et al., 2011, Constructing and deconstructing stem cell models of neurological disease, Neuron 70(4):626-44, the contents of each of which are incorporated by reference.

Stems cells may be converted into neurons 113, e.g., by direct lineage conversion. Conversion may include the use of lineage-specific transcription factors to induce the conversion of specific cell types from unrelated somatic cells. See, e.g., Davis-Dusenbery et al., 2014, How to make spinal neurons, Development 141:491; Graf, 2011, Historical origins of transdifferentiation and reprogramming, Cell Stem Cell 9:504-516, incorporated by reference. It has been shown that a set of neural lineage-specific transcription factors, or BAM factors, causes the conversion of fibroblasts into induced neuronal (iN) cells. Vierbuchen 2010 Nature 463:1035, incorporated by reference. MicroRNAs and additional pro-neuronal factors, including NeuroD1, may cooperate with or replace the BAM factors during conversion of human fibroblasts into neurons. See, for example, Ambasudhan et al., 2011, Direct reprogramming of adult human fibroblasts to functional neurons under defined conditions, Cell Stem Cell 9:113-118; Pang et al., 2011, Induction of human neuronal cells by defined transcription factors, Nature 476:220-223; also see Yoo et al., 2011, MicroRNA mediated conversion of human fibroblasts to neurons, Nature 476:228-231, the contents of each of which are incorporated by reference.

Differentiated cells such as neurons 113 may be dissociated and plated onto glass coverslips (e.g., to be the transparent bottom 112 of container 111) coated with poly-d-lysine and laminin. The neurons may be fed with a suitable media 138 such as an N2 medium. Suitable media are described in Son et al., 2011, Conversion of mouse and human fibroblasts into functional spinal neurons, Cell Stem Cell 9:205-218; Vierbuchen et al., 2010, Direct conversion of fibroblasts to functional neurons by defined factors, Nature 4 63:1035-1041; Kuo et al., 2003, Differentiation of monkey embryonic stem cells into neural lineages, Biology of Reproduction 68:1727-1735; and Wernig et al., 2002, Tau EGFP embryonic stem cells: an efficient tool for neuronal lineage selection and transplantation. J Neuroscience Res 69:918-24, each incorporated by reference. The differentiated cells are thus neurons, i.e., the neurons 113, living in the media 138. In preferred embodiments, the neurons 113 are transduced with optogenetic proteins. For example, a plasmid or a viral vector may be introduced (e.g., using electroporation or lipid nanoparticles) to introduce one or more genetically encoded proteins that function as optical actuators of, and/or optical reporters of, neural activity. See U.S. Pub. 2014/0295413, incorporated by reference.

To provide the sample 201 that includes one or more neurons 213 in media 238 that includes a plurality of pain mediators 215 associated with cancer pain, the pain mediators 215 are introduced. One method for introducing cancer pain mediators 215 is to co-culture the neurons 113 with cancer cells to yield the in vitro model. The neurons 113 may be cultured alone, or they may be cultured with fibrosarcoma cells (NCTC 2472). The neurons may be cultured with rapidly proliferating but non-malignant cells (fibroblasts). It may be found that including a cancer cell line modulates sensitivity to ATP-induced currents. Alternatively, neurons 113 can be cultured with the supernatant from an immortal cancer cell line like squamous cell carcinoma. In other embodiments, synovial fluid drawn from a joint affected by osteoarthritis is delivered to the neurons. In preferred embodiments, a pain mediator composition is prepared of its ingredients, selected pain mediators at selected concentrations. The pain mediators may provided as a cell-free composition, e.g., free in solution, and aliquoted into the media 138 to be introduced to the neurons 113 as a pain mediator composition.

FIG. 3 shows neurons 113 and a pain mediator composition that may be used to model an inflammatory environment of a joint affected by osteoarthritis. The neurons 113 include an optical reporter 811 of membrane potential such as a microbial rhodopsin or modified microbial rhodopsin with reduced ion pumping activity relative to wild type (e.g., Archaerhodopsin 3 D95N). Osteoarthritis (OA) is a type of joint disease that results from breakdown of joint cartilage and underlying bone. Articular cartilage lacks blood vessels and nerves. However, nerve endings land on the joint capsule (articular capsule). Nerves on the joint capsule include: Aa fibers (Golgi-type endings); A13 fibers (paciniform corpuscles and Ruffini terminals); and Ad & C fibers (nociceptors) form free nerve endings. Nociceptors also innervate joint fat pads.

Synovitis refers to an inflammation of the joint capsule and its lining, the synovial membrane, is common to both OA and rheumatoid arthritis (RA). The inflammation precedes, and likely plays a role in causing cartilage loss in normal disease progression. In OA, there are higher levels of blood plasma proteins in the synovial fluid possibly associated with the synovial membrane becoming permeable. It may be found that OA patients show increased inflammatory markers, increased in the blood but also greatly increased in the synovial fluid. Those inflammatory markers act as mediators of pain and inflammation. Certain mediators seem to play a consistent role as mediators of inflammatory pain.

Certain properties may be shared by the mediators and may serve as criteria for inclusion in a pain mediator composition. For example, if a molecule increases neural excitability and induces a hyperalgesic response in animals, then the molecule may be selected as a pain mediator and may be optionally interchanged with other pain mediators in the same category. Further, if the pain response of an animal knockout of a receptor associated with a molecule is not affected in the presence of the molecule, or if blocking such a receptor by pharmacological intervention decreases a pain response in an animal, then the molecule may be selected as a pain mediator.

For example, tumor necrosis factor alpha (TNF-a) is increased in synovial fluid, exhibits catabolic effects, and may have direct effect on nociceptors TNFR1/TNFR2, expressed in dorsal root ganglions (DRG). TNF-a may play a role in increased excitability of DRG neurons, and sensitization of C and Ad fibers. Nerve growth factor (NGF) is increased in synovial fluid in OA patients. TrkA and p75 are receptors for NGF, expressed in DRG. NGF exhibits increased excitability by direct effect on sensory neurons and effects on transient receptor potential (TRP) via phospholipase C (PLC). Injection of NGF in animals and humans produce hyperalgesia. Anti-NGF antibodies are efficacious in OA pain. Interleukins (ILs) represent a class of chemical mediators of pain or inflammation. IL-1b is increased in synovial fluid, involved in cartilage degradation, is an inducer of cytokines and direct effect on nociceptors. IL-1RI, expressed in DRG, is associated with increased excitability, and excites C but not Ad fibers. IL-1RI leads to hyperalgesia after injection and there is efficacy by inhibition of IL-1RI. The pain signaling molecule IL-6, the ligand for IL-6Rα, is found at increased concentration in the synovial fluid of OA patients. IL-6Rα/gp130, expressed in DRG, is associated with increased excitability and sensitization of C fibers to mechanical stimulus. IL-17 is increased in synovial fluid, is a mediator of immunity and inflammation, and is associated with arthritic disorders. IL-17RA is expressed in DRG and is associated with increased excitability of DRGs. Further, osteoarthritis is associated with a decrease in local pH during osteoclastic bone resorption, inflammation and tissue hypoxia. The ASIC and TRPV1 ion channel receptors sense pH changes, increase excitability of DRG neurons, and are associated with hyperalgesia. Blocking these channels is associated with analgesia. For those reasons, TNF-a, NGF, IL-1b, IL-1RI, IL-6, IL-6Rα/gp130, IL-17j, ASIC3 and TRPV1 are all good candidates for inclusion in an osteoarthritis pain mediator composition.

Further pain mediator candidates for an osteoarthritis pain mediator compositions include: PGE2 (increased in synovial fluid; the associated EP1 and its receptors are expressed in DRGs); CCL2/MCP-1 (ligand and receptor elevated in DRGs in experimental OA; produce direct effects on nociceptors and also recruitment of macrophages); and CCR2 (expressed in DRG; increases Ca2+ signaling in cultured DRGs and increases excitability of intact DRGs in vivo; CCR2 null mice show reduced pain behavior in OA model). Additional molecules that may be pain mediators include peptides (VIP, BK, SP, CGRP) which may be expressed in nociceptors and may be released on nociceptor activation. Further molecules that may be pain mediators include Damage-Associated Molecular Patterns (DAMPs) (present in OA joints); TLR-4 (expressed in DRG; a primary receptor activated by two types of DAMPS); alarmins; plasma proteins; matrix metalloproteinases (MMPs) (1,3,9,13) (MMPs are expressed in the OA joint; may function as collagenases); MMP-3,-9 (degrade non-collagen matrix components of joints); and FGF-2, VEGF, and EGF (increased in synovial fluid in OA; growth factors involved in cartilage homeostasis (FGF-2) and angiogenesis (VEGF, EGF)). Any of the foregoing may be a useful molecule to include in a pain mediator composition. See FIG. 25 for a list of pain mediators included in an osteoarthritis inflammatory pain mediator composition according to certain embodiments. The pain mediators included in this osteoarthritis inflammatory pain mediator composition were selected in accordance with the methods of the disclosure.

FIG. 4 illustrates neurons 113 and a plurality of pain mediators that may be used to model a tumor microenvironment. The neurons 113 include an optical reporter 811 of membrane potential such as a microbial rhodopsin or modified microbial rhodopsin with reduced ion pumping activity relative to wild type (e.g., Archaerhodopsin 3 D95N). Cancer pain may be caused by tumor-secreted molecules without being caused, necessarily, by local pressure or mechanical damage. Pain mediators to be included in a cancer pain mediator composition may include IL-6 (NSAIDs downregulate release); TNFa (NSAIDs downregulate release); NGF; trypsin, H+; P2X; and others. See Example 1 (infra) and Schmidt, 2014, The Neurobiology of Cancer Pain, Neuroscientist 20(5):546-62, incorporated by reference. Cancer pain mediators have historically been imperfect drug targets, subject to extensive development with limited success. The complex interplay of these components mediate pain; therefore, blocking one component in isolation will likely have limited efficacy.

Each molecule selected for inclusion in a cancer pain mediator composition should satisfy certain criteria of the disclosure. For example, the molecule should be detected at increased levels in or near tumors in people, preferably across more than one type of cancer. The associated receptor should also be expressed in sensory neurons. When injected in rodents, the molecule should cause local pain. Ideally, a pharmacological blocker of the associated receptor will alleviate pain in the injected rodent. Additionally, a mouse knockout of the receptor should not experience pain upon injection of the molecule.

To give an example, a protease such as trypsin should satisfy those criteria. PAR2 is a G-protein coupled receptor (GPCR) that modulates inflammatory responses and acts as a sensor for proteolytic enzymes generated during infection. PAR2 is highly expressed in DRGs. Dialysis of tongue tumor supernatant followed by mass spectrometry has shown an increased trypsin level compared to a control from the healthy side of the tongue. Proteases are universal in tumors as they are necessary to expand into tissue and for eventual metastasis. Trypsin is present in breast, colon, and oral cancers. It is suspected that a protease such as trypsin cleaves accessible portions of a receptor such as PAR2, thereby stopping the ability of PAR2 to modulate an inflammatory response. When PAR2 is cleaved by a protease, the remaining tail drives specific activation of the receptor. PAR2 and TrpV4 work together for mechanical hyperalgesia. This insight suggests that proteases may be a useful ingredient of a cancer pain mediator composition.

Other mediators that are found in the tumor environment include opioid peptides; endothelin 1 (found in many types of cancers); ATP via P2X3 receptor; NGF (NGF sequestration with an antibody appears effective in reversing cancer pain in preclinical cancer models; BDNF (not strongly correlated with pain); bradykinin; TNF-a; legumain (elevated both in supernatant and ground up tumor); and cathepsin S. Growth factors, like interleukins and proteases, are another example of a type of mediator frequently associated with cancer pain. NGF is elevated in human cancer and mouse models of oral cancer. Immunohistochemistry shows strong NGF immunoreactivity in a representative tissue biopsy from an oral cancer patient. In contrast, normal oral epithelium shows faint NGF immunoreactivity. Real-time PCR quantification of NGF mRNA in oral SCC biopsies shows a large increase over normal tissue. Evidence has shown that NGF protein is elevated in tissue associated with a tumor. See Example 1, infra, for further discussion.

FIG. 5 is a taxonomy of pain mediators. At a highest level, the mediators are unified in being those elements that have been shown or understood to participate in causing a human sensation of pain in response to cancer, an inflammatory condition, or other disease condition. Those mediators may be divided into proteins, other chemical species, and phenomenological stimulus such as tissue trauma. The other molecular species includes things other than proteins synthesized by the body, things such as fragments of extra cellular matrix (ECM), protons/acidic pH, and any other molecules such as steroids. Any of the mediators may also be categorized in other ways such as nociceptive triggers or as modulators of sensory processing.

In fact, it may be beneficial to use this level of taxonomy to define a pain mediator composition, such that the composition includes at least three different categories of mediators, each independently selected from an interleukin, a growth factor, a protease, a tumor necrosis factor, and protons. The pain mediator composition may include a plurality of further mediators selected from the group consisting of histamine, serotonin, ATP, bradykinin, prostaglandin E2, and endothelin-1. An effective pain mediator composition may be found to preferably include multiple (e.g., 3, 4, 5 or more) unique ingredients selected from these protein classes and other pain mediators.

For osteoarthritis pain applications, the selected pain mediators in a pain mediator composition may include proteins found to have increased expression in synovial fluid of osteoarthritis subjects relative to blood or serum of a healthy subject. For cancer pain applications, the selected pain mediators in a cancer pain mediator composition may preferably include proteins found to have increased expression in a tumor environment relative to blood or serum of a tumor-free subject.

At a specific level, the pain mediators may include several of TNF, IL, PGE2, substance P and a growth factor; a signaling mediator selected from the group consisting of NFkB, ERK1/2, p38, JK, PKCdelta, TLRs, beta-catenin, Gli1, Ptch, HHIP, HIF-2alpha, iNOS, and RUNX2; and a protease. For example, the plurality of pain mediators may include at least five of TNF-a; IL-1b; IL-6; IL-1; IL-15; IL-16; prostaglandin; fragments of extra-cellular matrix; trypsin; ATP; H+; neurturin; GDNF; and BDNF.

The disclosure includes pain mediator compositions with pain mediators at selected concentrations. Concentrations may be selected to approximate the relative concentrations of such mediators in the affected tissue, to ensure that each ingredient is playing a chemical and functional role in the composition, or both. For example, synovial fluid or tumor infiltrate may be analyzed quantitatively to determine relative amounts of constituent molecules. In another example, prospective mediators may be plated and assayed in various concentrations (e.g., as shown in FIG. 12) both alone and among a full suite of prospective mediators. Each aliquot of such an assay can be tested to determine a contribution by the subject mediator to an effect on neural activity, and concentrations in which any ingredient contributes no effect may be avoided. It may be preferable (looking at the plate shown in FIG. 12) to select, for each ingredient, the lowest concentration at which that ingredient contributes demonstrable effect to neural activity. In one exemplary embodiment, the ingredients have the concentrations given in FIG. 25, so selected at least in part for effect that was measured as indicated by the text entries in that table.

The described pain mediator compositions are used to provide a sample that includes one or more neurons in media which includes a pain mediator composition. Methods of the disclosure include measuring an effect of a test compound in the presence of a pain mediator composition on neural activity in the sample. In certain embodiments, during the measuring, at least one of the neurons lives in the media within a measuring instrument that includes a control unit operable to control at least temperature or pressure above the liquid media. As will be discussed in greater detail below, the neurons may optionally include optogenetic reporters and/or actuators and may be stimulated and measured using light, e.g., by using a light microscopy instrument.

The measuring instrument may include an optical microscopy system operable to optically trigger an action potential in at least a first neuron in the media, to optically excite a fluorescent reporter of action potential in the neurons, and optically detect an action potential reported by the reporter.

FIG. 6 shows a microscope 600 with high spatial and temporal resolution for imaging dynamic processes. The microscope 600 may be used in conjunction with fluorescence imaging wherein the fluorescence may be mediated by voltage-indicating proteins (e.g., microbial rhodopsins) to measure the electrical properties of cells. The microscope 600 may also be used with light-sensitive activators to allow selective, variable intensity activation of cells. Components include a large FOV, high-resolution imaging subsystem; an illumination subsystem; an optional activation subsystem; and a sample positioning and environmental control subsystem. The microscope 600 may be used to image and illuminate the neurons 113 that express optogenetic proteins. The microscope 600 includes a sample enclosure 51 serviced by an environmental control system. A top platform 55 is supported above side walls and base platform 61. The environmental control system 21 is part of the overall sample handling system. This provides control over humidity, temperature, gas, and sample positioning.

The uprights 59, top platform 55, and base platform 61 cooperate to define a chassis for supporting the illumination and imaging subsystems. Additionally, the top platform 55 and base platform 61 may include mounting flanges (e.g., with threaded holes) to which side walls can be affixed, thus provided a dark enclosure within which the optical elements of the microscope can be housed and operate. A sample positioning system is installed in the top platform 55. The sample positioning system can be used to adjust the object region 609 up, down, left, right, forward, and backwards. It can also adjust the tilt of the object region and it can rotate the sample.

FIG. 7 illustrates an embodiment of how the illumination subsystem 631, the imaging subsystem 691, and the activation subsystem 641 are disposed within device 600. For the illumination subsystem 631, an illumination light source 627 generates illumination light 635 which via optical elements 633 and 637 (e.g., stepper/translator mirrors) impinges on a prism 625.

For the activation subsystem 641, activation light 644 is reflected off of optical element 653 (e.g., a dichroic mirror) and then passed through the objective unit 629, after which it impinges upon a sample in sample stage 609. In a preferred embodiment, the optical element 653 is a spatial light modulator such as a digital micromirror device (DMD), e.g., as available from Texas Instruments. In the imaging subsystem 691, image light 693 passes from the sample stage 609 through the objective unit 629 and through the optical element 653 to a camera or light detector 697. A base platform 61 is supported by supports 69 on a lower platform 75, which employs leveling feet 77. The feet 77 can be used to level the sample stage 609 (shown in FIG. 6). Because the open-stage microscope provides a sample stage configured to contain samples including aqueous solutions, it is important to ensure the stage is level. The feet can include a screw portion engaged with a threaded hole in the lower platform 75. The feet 77 can be adjusted by turning their respective screws. The supports 69 are slightly inset from the location of the feet 77, thereby providing access for the feet 77 to be adjusted by a human operator. The base platform 61 and lower platform 75 should be substantially flat and made from a resilient material such as aluminum or steel. The feet 77 and supports 69 should likewise be manufactured from a resilient material capable of withstanding the weight of the microscope and all of its components.

FIG. 7 shows details of the imaging subsystem 691. Preferably the imaging light 635 after passing through prism 625 passes through an imaging tube lens 659 and to a light detector/camera 697. The imaging subsystem 691 may include a low magnification, large FOV, high NA tube lens 659; and an image sensor 697, as shown in FIG. 7. In some embodiments, the imaging light passes through a dichroic mirror 653 that is part of the activation subsystem. The sample stage 609 is supported above an objective lens unit 629. Components of an illumination subsystem are also shown mounted to uprights 59. The illumination subsystem includes an illumination light source 627 (e.g., a red diode laser bar), a mirror 633 and an adjustable mirror 637, which can be adjusted manually or by way of a stepper or translation motor. The sample 201 may be placed in the object region 609 of the microscope 600, wherein the sample dish 111 comprises a transparent bottom portion 112 and contains a biological sample (e.g., the neurons 113) in an aqueous medium 138. The microscope 600 is used for illuminating the sample 201 from beneath via an illumination light 635 passing through a prism 625 disposed underneath the transparent bottom portion, whereby the prism 625 imparts near total internal reflection on the illumination light; and imaging 521 the biological sample through the transparent bottom portion using an imaging subsystem 691 of the microscope 600, the imaging subsystem 691 comprising an objective lens unit 629 disposed beneath the sample dish 611 and an image capture device 697. Preferably, the prism and transparent bottom portion are coupled by a low auto-fluorescence index matching fluid; and the prism, the index matching fluid, and the transparent bottom portion have a common index of reflection. The sample dish can provide access to the sample from above. The method 501 may include using an environmental control subsystem to control environmental conditions above the aqueous medium to maintain living cells 613 in the aqueous medium. As discussed elsewhere herein, the environmental control subsystem may control humidity, temperature, or gas of the sample region. The transparent bottom portion of the sample dish may be provided by a coverslip mounted to a surrounding dish structure.

Between the objective lens and the object region 609 is a prism 625. In a preferred embodiment, the prism 625 has a trapezoidal cross-section with beveled edges. The prism 625 is configured to impart near total internal reflection (TIR) on an illumination beam passing therethrough. Near-TIR excitation serves to limit the production of unwanted fluorescence. The prism and object region 609 are configured such that a user can place index matching fluid between those two elements. In embodiments that include index matching fluid, the near-TIR is exhibited at boundary between the glass coverslip and the media 138 containing the sample.

The objective lens 629 preferably has a numerical aperture that is high for lenses of low magnification, for example 0.4, 0.5, 0.6, 0.7, or 0.85. Examples of suitable objective lenses 629 include model number MVPLAPO 2XC manufactured by Olympus Corporation.

The tube lens 659 may be a standard, 3″ diameter plano-convex lens with focal length of 100 mm or it may comprise several elements with a similar equivalent focal length. The choice of 100 mm focal length of the tube lens 659 is matched to the reference tube length of the MVPLAPO 2XC objective (resulting in a magnification of 2×). A different focal length may be selected for other magnification. Additional lenses may be added to reduce aberrations.

The image sensor 697 has sufficient resolution and bandwidth to record signals over the field of view at the desired frame rate. One example of a suitable image detector is a scientific CMOS camera such as the CMOS camera sold under the trademark FLASH 6.0 by Hamamatsu Corporation. Values and specifications of the imaging system components may be adjusted to optimize performance for many circumstances such as a different objective lens, field of view, image sensor, fluorophore, frame rate, or tube lens. The imaging light path may include other optical elements to improve performance.

The system 600 may further comprise additional components to aid in observing and imaging a sample. For example, it may comprise a camera such as a CMOS camera, for obtaining an image from the imaging optical path. The system may also have a mechanism for adjusting the object region, manually or via computer control, by tilting or translation. See WO 2016/187543 A1.

Fluorescent reporters such as Arch 3, Arch 3 D95N, QuasAr1, QuasAr2 and QuasAr3 use light in order to fluoresce. The illumination subsystem emits light at high wattage or high intensity. The use of near-TIRF illumination exposes only a small portion of the sample to the illumination light, thereby reducing excitation of the culture medium or other components of the device. Additionally, the microscope is configured to provide illumination light that is distinct from imaging light. Optical filters in the imaging subsystem filter out illumination light, removing unwanted fluorescence from the image.

The near-TIRF microscope is configured to optically characterize the dynamic properties of the neurons 113. The microscope can provide a field of view sufficient to capture tens or hundreds of cells. The microscope and associated computer system provides a very fast image acquisition rate on the order of 1 kilohertz, which corresponds to a very short exposure time on the order of 1 millisecond, thereby making it possible to record the rapid changes that occur in the neurons 113. The microscope can therefore acquire fluorescent images using the recited optics over a substantially shorter time period than prior art microscopes.

Samples useful with the near-TIRF microscope include the neurons 113 expressing an optical activator of electrical activity and an optical reporter of electrical activity. The sample may be configured such that a first cell expresses the activator and a second cell expresses the reporter. The microscope can activate the light-sensitive activator protein with an activation beam to cause a conformational change in the protein, thereby initiating a change in membrane potential in the cell. The result is that the cell “fires,” i.e., an action potential propagates in the electrically-active cell. The microscope can simultaneously illuminate a fluorescent optical reporter protein with an illumination beam that is spectrally distinct from the activation beam, causing the reporter to fluoresce. The imaging subsystem of the microscope can measure the fluorescence emitted by the reporter to measure corresponding changes in membrane potential.

In some embodiments, the neurons 113 include actuator that initiates an action potential in response to incoming light, a reporter that emits light in response to an action potential, or both. Measuring neural activity may include measuring an optical signal emitted by a neuron of the one or more neurons.

FIG. 8 illustrates an optical actuator 803 of neural activity and an optical reporter 811 of neural activity. Preferably, the one or more of the one or more neurons 113 include optical actuators of neural activity, optical reporters of neural activity, or both (the figure is a close-up of a portion of a membrane of one of the neurons 113). See U.S. Pat. No. 9,518,103.

The microscope 600 can be used to observe fluorescent indicators that are sensitive to specific physical properties of their environment such as calcium ion concentration reported by a calcium indicator 817 or membrane potential reported by the optical reporter 811. The time-varying signal produced by these indicators is repeatedly measured to chart action potentials in the neurons 113. One example of an environmentally sensitive fluorescent indicator (e.g., the optical reporter 811) for use with the present disclosure is the archaerhodopsin-based protein QuasAr2, which is excited by red light and produces a signal that varies in intensity as a function of cellular membrane potential. QuasAr2 can be introduced into cells using genetic engineering techniques e.g., via a plasmid or viral vector.

In addition to measuring the action potentials, a microscope 600 can be used to optically activate the optical actuator 803. The methods of this disclosure can use voltage-indicating proteins such as those disclosed in U.S. Patent Publication 2014/0295413, filed Jun. 12, 2014, the entire contents of which are incorporated herein by reference. Using light-controlled activators, stimulus can be applied to entire samples, selected regions, or individual cells by varying the illumination pattern. One example of a light-controlled activator (e.g., the optical actuator 803) is the channelrhodopsin protein CheRiff, which produces a current of increasing magnitude roughly in proportion to the intensity of blue light falling on it. In one study, CheRiff generated a current of about 1 nA in whole cells expressing the protein when illuminated by about 22 mW/cm2 of blue light.

Optically modulated activators can be combined with fluorescent indicators to enable all-optical characterization of specific cell traits such as excitability. For example, a channelrhodopsin such as CheRiff is combined with a fluorescent indicator such as QuasAr2. The microscope provides different wavelengths of light to illuminate and activate the reporter and activator proteins, respectively, allowing membrane potential to be measured at the same time that action potentials are initiated by light. The neurons may further optionally include a calcium indicator 817 such as GCaMP6f. The optical actuator 803 may be linked to a fluorescent protein 825 (e.g., eGFP).

Measuring neural activity may include recording an action potential and storing a representation of an action potential waveform.

FIG. 9 illustrates the recording of action potential waveforms from neurons with optical reporters of electrical activity using an instrument of the disclosure. In all parts of the figure, the x axis is the same and is time. The center portion labeled “blue stim” represents a power of light (blue) that was shined onto the neurons 113 to trigger action potentials via the optogenetic actuators. The traces above the center portion show a potential difference measured across a cell membrane using an optical reporter (e.g., a modified microbial rhodopsin such as QuasAr) of neural activity. Thus the traces represent action potentials being recorded as the neurons 113 fire in response to the blue light stimulation. Individual cells were measured under buffer only (no pain mediator composition), after 30 minutes of exposure to a pain mediator composition, and after 48 hours of exposure to the composition, as the labels so indicate.

The bottom panel gives the aggregate, average pattern of AP spikes for a large number of neurons under the indicated conditions. It can be seen that under acute exposure (30 min), the neural phenotype is characterized by more spiking cells, faster spiking, and broadened action potentials. Under chronic exposure, the neural phenotype is characterized by highly excitable single-spiking cells. This suggests that the pain mediator composition induces drastic changes in excitability and action potential shape, a measurable phenotype that can be used to test effect of drugs.

FIG. 10 shows various action potentials measured using an instrument and sample 201 of the disclosure. As shown, phenotypical features that may be observed or measured include spike timing, spike shape, excitability, and average spike waveform. The “excitability” panel illustrates one successful and interesting test of the disclosure. Pre-intervention, the neurons 113 were characterized by a certain demonstrable spike rate in Hz. Upon acute introduction of a pain mediator composition, that spike rate increased significantly. After treatment with 1 micro-molar of a test compound, the spike rate decreased to a value closer to baseline. Measuring neural activity includes recording an action potential and storing a representation of an action potential waveform.

For ease of reference, the measured results can be displayed as a radar plot.

FIG. 11 shows a radar plot of action potentials. The darkest line around the plot is a firing pattern of a neuron in media in the absence of a pain mediator composition. The light line (showing a high number of spikes) is an average firing pattern of a neuron when the media includes a pain mediator composition. The medium darkness line around the plot shows the neural activity after test compound was introduced to the neuron in the presence of the pain mediator compositions.

The radar plot is convenient for the inspection of differences between different aspects of action potentials, i.e., changes in neural activity, and thus an effect of a test compound on neural activity. The radar plot aids in comparing the action potentials by, for example, determining differences between one or more of spike width, after-hyperpolarization depth, stimulation intensity at maximum spike rate, maximum spike rate, and number of spikes under a fixed period.

Such a comparison may be useful in comparing the effect of a test compound on neural activity in the presence of a pain mediator composition to an effect of the mediators on neural activity in the absence of the test compound by comparing action potentials of neurons under the respective conditions.

FIG. 12 shows a plating strategy that may be used in the creation of a pain mediator composition or may be employed when using a pain mediator composition in a screening assay. The creation of the pain mediator composition may include selection of the mediators (e.g., based on their presence in or near a target tissue associated with a pain response) and determining the concentration of each mediator (e.g., such that (i) the pain mediators are combined in relative concentrations that approximate the relative concentrations of such mediators in the target tissue; (ii) the concentrations ensure that each ingredient is playing a chemical and functional role; or (iii) both). As shown in FIG. 12, in the left five columns of a 96 well plate, a pain mediator is introduced into five adjacent wells in increasing concentration, along with the other ingredients of a pain mediator composition. Continuing to read across a row, the next well is the complete pain mediator composition (“S”). The next well is the vehicle (“V”) alone (most drugs will be delivered with a vehicle such as DMSO or saline). The next five wells are the mediator alone, in increasing concentration. Every well includes neurons 113. Each row is dedicated to one constituent ingredient of the composition (in this embodiment, NGF, ET-1, TNF-a, IL-6, bradykinin, ATP, pH, and trypsin). Such a plate may be used to obtain a baseline firing phenotype for neurons exposed to a plurality of pain mediators, and also to assay for a relative contribution to the pain phenotype of each of the constituent mediators in order to select the concentration of each pain mediator in the pain mediator composition.

Selecting the concentration of each ingredient of the pain mediator composition may include one or more of any suitable method or step. For example, the concentrations may be determined by an assay of or from affected tissue. Additionally or alternatively, the concentrations may be determined by testing the concentration of each single component in various (e.g., ascending) concentrations and then as part of a mixture as is shown in FIG. 12. It may be beneficial to model the contribution of each ingredient at different concentrations, taken experimentally from an assay such as shown in FIG. 12, and use a mathematical model such as a principal component analysis, a maximum likelihood model, or similar model to determine a set of concentrations at which the contribution from each ingredient is optimized.

FIG. 13 gives results from another instrument run, but with a test compound being tested (axes and labels and similar as for FIG. 9). For each of cell 1, cell 2, and cell 3, the top trace represents action potentials measured before intervention with a pain mediator composition or a test compound, the middle trace is taken after intervention with the pain mediator composition, and the bottom trace is measured after the neurons 113 are exposed to the pain mediator composition plus a test compound. The “pre-intervention” traces represent a baseline firing phenotype obtained for neurons free of any pain mediators, the traces labelled “inflammatory soup” represent a baseline firing phenotype for neurons exposed to the pain mediator composition. In the figure, certain traces are labeled “1 μM TTX” and those traces represent the effect of a test compound on neural activity and thus on a pain sensation that would be experienced by a human.

The presented data show steps of a systematic process for testing a drug. Those steps include providing a sample 201 that includes neurons 113 in media 138 that includes a plurality of pain mediators 105. A test compound (“TTX”) is introduced, and an effect of a test compound is measured in the presence of the pain mediator composition 105 on neural activity in the sample 201.

U.S. Pub. 2018/0031553 and U.S. Pat. No. 9,207,237 are incorporated by reference. WO 2016/187543 is incorporated by reference. U.S. Pat. No. 9,518,103 is incorporated by reference.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

### EXAMPLES

### Example 1: Cancer Pain Mediator Composition

Putative mediators for inclusion in a cancer pain mediator composition were analyzed according the criteria of the invention. The criteria include (i.) detection of the mediator around tumors at increased levels relative to the levels in healthy tissue; (ii.) the associated receptor for the mediator is expressed in neurons used in the screening panels; and (iii.) the mediator causes local pain in animal models. Other optional criteria include: (iv.) a pharmacological block of the mediator-associated receptor alleviates pain in animal models; and (v.) a genetically modified animal model that does not express receptors associated with a specific mediator does not experience pain when exposed to the mediator.

Nerve growth factor (NGF) is elevated in human and mouse models of oral cancer. Tumors and constituent cells have been found to secrete NGF in multiple cancer types. DRG neurons express TrkA/TrkB receptors associated with NGF. NGF sequestration using anti-NGF antibody has been found effective in reversing cancer pain in preclinical cancer models. Accordingly, NGF meets at least criteria i.-iv. and, therefore, may be selected for inclusion in a cancer pain mediator composition. Because increased NGF levels are associated with multiple cancer types, NGF may be included in a general cancer pain mediator composition because its effects are not limited to a particular type of cancer.

Increased endothelin 1 (ET-1) levels have been associated with multiple cancer types including prostate, breast, colon, hepatocellular, pancreatic, endometrial, lung, and oral cancer. DRG neurons express ETAR and ETBR receptors are associated with ET-1 induced pain. ET-1 strongly resembles sarafotoxins found in snake venom, and subcutaneous injection of ET-1 leads to local sensitization in animal models (measured by paw withdrawal threshold). Accordingly, ET-1 meets at least criteria i.-iii. and may be selected for inclusion in a cancer pain mediator composition including general cancer pain mediator compositions due to its association with multiple cancer types.

Tumor necrosis factor alpha (TNF-a) is an inflammatory cytokine found to be elevated around tumors. TNF-a triggers a signal-transduction cascade, modulates transcription, and is generally pro-apoptotic. DRG neurons express TNFR1 and TNFR2 receptors associated with TNF-a-induced pain. TNF-a has been found to cause pain when injected in rats and anti-TNF-a antibody has been found to relieve TNF-a-associated pain in humans. Accordingly, TNF-a meets at least criteria i.-iv. and may be selected for inclusion in a cancer pain mediator composition.

Interleukin 6 (IL-6) is an inflammatory cytokine found to be elevated around tumors. Like TNF-a, it triggers signal-transduction cascade and modulates transcription. DRG neurons express IL-6Ra and gp130 receptors associated with IL-6-induce pain. IL-6 has been found to causes local pain when injected in rats. Accordingly, IL-6 meets at least criteria i.-iii. and may be selected for inclusion in a cancer pain mediator composition.

Bradykinin (BK) is secreted by melanoma, activates B receptors, and causes cancer pain. The B1 receptor has been implicated in bone cancer pain. B1 and B2 G-protein coupled receptors, associated with BK induced pain, are expressed by DRG neurons. BK injection in mice has been found to cause local pain and B-receptor blockers have been shown to provide pain relief. Accordingly, BK meets at least criteria i.-iv. and may be selected for inclusion in a cancer pain mediator composition.

Many cancers upregulate expression of the purinoceptor for ATP, P2X3, so that released ATP causes greater depolarizations and pain. Blocking the P2X3 receptors has reduced pain in mouse models. Luciferease measurements have also shown ATP excretion from tumors in vivo and ATP injection in humans has been shown to cause increased pain sensitivity. P2X3 receptors are expressed by DRG neurons. Accordingly, ATP meets at least criteria i.-iv. and may be selected for inclusion in a cancer pain mediator composition.

Many tumors are known to increase acidity in forming their cancer microenvironment and expression levels of the capsaicin receptor TRPV1 and acid-sensing ion channels (ASICS) have been found to be elevated near tumors. The increased ASICs expression is in response to the acidic environment. The ASICS and TRPV1 channels are expressed by DRG neurons and acidosis is a known cause of pain. While the healthy human body typically has a pH of about 7.4, pH in cancer microenvironments is found to be around about 6.5 to about 6.8. Because H+ meets at least criteria i.-iii., compositions of the invention may be slightly acidic to approximate the cancer microenvironment in reproducing cancer-associated pain.

Protease-activated receptor 2 (PAR2) is a G-protein coupled receptor that modulates inflammatory responses by acting as a sensor for proteolytic enzymes generated during infection and is expressed by DRG neurons. Trypsin and other proteases, released by tumors as they expand into tissues, cleave the extracellular tail of PAR2, and the remaining stub activates the signaling protein. Increased trypsin levels are found at least in breast, colon, and oral cancers. Pharmacological or genetic blocking of PAR2 has been found to reduce cancer pain in a mouse model. Accordingly, trypsin meets at least criteria i.-v. and may be selected for inclusion in a cancer pain mediator composition including general cancer pain mediator compositions due to its association with multiple cancer types.

### Example 2: Tool Compounds

FIGS. 14-16 gives results from the use of a tool compound. A tool compound may be a compound, biologic or chemical species that is understood to have sufficient potency, selectivity, cell permeability, or bioavailability to be used for the validation of a hypothesis of analgesia for a test compound in methods of the disclosure. Thus, measuring results using a tool compound of known effect can provide a phenotype to use as a standard when looking at a phenotype associated with treatment of a sample 201 with a test compound. FIG. 14 shows spike frequency of neurons before and after treatment with a pain mediator composition.

FIG. 15 shows the treatment of the neurons 113 with A-803467, a selective blocker of Nav1.8.

FIG. 16 shows treatment with PF-05089771, a selective blocker of Nav1.7.

In FIGS. 15 and 16, the radar plots show that those tool compounds of known mechanism function to restore the neural phenotype to a phenotype approximating the neurons 113 absent the pain mediator composition. Thus tool compounds may be valuable for establishing a criteria against which to compare an effect of test compounds.

FIGS. 17-20 give concentration response curves (CRC) for several tool compounds (duloxetine, flupirtine, PF-05089771, and ProTx-II).

FIG. 17 gives a concentration response curve CRC for duloxetine.

FIG. 18 gives a CRC for flupirtine.

FIG. 19 gives a CRC for PF-05089771.

FIG. 20 gives a CRC for ProTx-II.

Tool compounds establish standards by which to measure test compound and also show that robust CRC's can be measured with many drugs and multiple mechanisms

### Example 3: Optogenetic Methods

FIG. 21 diagrams an optogenetic method 2101 of testing a drug. The method 2101 includes providing 2105 a sample that includes one or more neurons in media comprising a pain mediator composition. The neurons include and express an optical reporter of neural activity, preferably in the form of a protein that emits light in response to an action potential. One suitable protein is a microbial rhodopsin such as Archaerhodopsin 3 (Arch) or a modified version thereof. The method may include shining light onto the sample to excite 2109 Arch. The neurons may also include an optical actuator of neural activity, i.e., a protein that triggers an action potential when hit with light of a certain wavelength. One such protein may be a light-gated ion channel such as an algal channelrhodopsin to function as the actuator. The method 2101 includes shining light 2113 onto the actuator, which causes an action potential. Due to the Arch, the action potential causes the neurons to illuminate, i.e., to directly give off light. Light originates at the Arch and travels from the neurons 113.

The method 2101 includes detecting 2121 the neural illumination. That neural illumination has a power that changes over time consistent with the action potential (AP) and thus a representation of the detected light represents a waveform of the AP. The method 2101 may include storing 2135 (e.g., in a tangible, non-transitory computer-readable memory) the AP waveform.

### Example 4: Assay of Pain Mediators

Pain mediators may be assayed to identify those present in representative tissue environments.

FIG. 22 shows certain pain mediators and the concentrations assayed in various replicates from samples of normal serum from a health patient, serum from a patient with osteoarthritis, and synovial fluid from a patient with osteoarthritis.

The tree diagram on the right of the figure shows a closeness of covariance in concentration. Thus, for example, MIG and IP-10 vary across normal sera and synovial fluid tightly with each other, while MIG and VEGF do not exhibit such a significant co-variance. The disclosure posits that a useful pain mediator composition to induce inflammatory pain may include one representative from each monophyletic group on the tree (at any level of the taxonomy), and that the tree represents a taxonomy of classes of mediators for inclusion. See Sokolove, 2013, Role of inflammation in the pathogenesis of osteoarthritis: latest findings and interpretations, Ther Adv Musculoskel Dis 5(2):77-94, incorporated by reference.

### Example 5: High-Throughput Assays

FIG. 23 shows a plating for heterologous screening. In the left 10 columns of a plate, a mediator is introduced into ten adjacent wells in decreasing concentration, along with all of the other ingredients of a pain mediator composition. The next ten wells are the mediator alone, again in decreasing concentration. Continuing to read across a row, the wells include tool compound 1, tool compound 2, vehicle alone, and a treatment. Such a plate may be used to measure a test drug with neurons exposed to a pain mediator composition. (T=1000 nM TTX; V=vehicle (0.5% DMSO); A=50 μM amitriptyline; R=100 μM veratridine).

All “V” and empty wells should contain vehicle −0.5% DMSO in Tyrode's.stock.

The plates are used to screen in duplicate (˜5000 compounds).

Test compounds may be screened over the wells of a plate such as the one depicted here. Radar plots obtained for each well (over a suitable number of replicates, with multiple action potentials averaged, optionally) may be compared to those radar plots from tool compounds known to exhibit suitable properties or elicit a desirable phenotype in vivo. It may be preferable to establish a hit threshold (e.g., as a % conformance to one or more measures from an action potential associated with a tool compound, e.g., 85% similar to). In some embodiments, a test compound is deemed a “hit” or active compound when treatment of the sample 203 (that includes one or more neurons 113 in media 138 comprising a pain mediator composition 105) with a test compound yields an effect on neural activity (e.g., in the form of an action potential) that can be measured in the sample in the presence of the pain mediator composition and has been established to meet the hit threshold. Thus, methods 101 of the disclosure are useful for testing compounds ex vivo to discover their in vivo analgesic properties.

### Example 6: One Embodiment of Cancer Pain Mediator Composition

A plurality of pain mediators 105 can be described as a pain mediator composition and may be used to model cancer pain, pain associated with an inflammatory condition such as osteoarthritis, or pain arising from other disease conditions.

FIG. 24 shows an exemplary method 201 for preparing a cancer pain mediator composition. Criteria for inclusion in the cancer pain mediator composition of the invention may include: (i.) detection of the mediator in or near tumors at increased levels relative to the detected levels in healthy tissue 2403; (ii.) the associated receptor for the mediator is expressed in neurons used in the screening panels 2405; and (iii.) the mediator causes local pain in animal models 2407. Other optional criteria include: (iv.) a pharmacological block of the mediator-associated receptor alleviates pain in animal models 2409; and (v.) a genetically modified animal model that does not express receptors associated with a specific mediator does not experience pain when exposed to the mediator 2411. A cancer pain mediator composition may be prepared comprising molecules that meet the above criteria. Exemplary application of the criteria is given below in the examples.

In order to characterize the cancer microenvironment, samples may be taken from the area in or near a tumor in a cancer patient or may be taken from the supernatant of tumor cells cultured in vitro. The molecular composition of the samples may be determined by mass spectrometry or other known means. The composition of cancer-associated samples may be compared to the composition of samples from healthy cells and tissue in order to identify molecules present at increased levels in the cancer-associated samples relative to the healthy samples. In addition to identifying specific molecules, characteristics such as acidity may be determined in both tumor-associated and healthy samples for more accurate formulation of a cancer pain mediator composition.

In creating cancer pain mediator compositions that are specific to a type of cancer, rather than generally applicable to any cancer, analysis should be made of samples from the specific cancer type to be modeled (e.g., prostate, breast, colon, hepatocellular, pancreatic, endometrial, lung, or oral). In creating general or pan-cancer pain mediator compositions, mediators may be prioritized based on the number of cancer types in which the mediator was found at increased levels where priority is given to mediators found in multiple cancer types. Mediators may also be prioritized based on the amount of the increase in tumor-associated samples over healthy samples, where a higher percentage increase carries more weight for inclusion in the cocktail.

Because compositions of the invention are envisioned as being used in, among other applications, in vitro assays for observing neuronal responses to the composition and putative therapies, the neurons used in the assays should express a receptor associated with any mediator included in the composition. Otherwise, the direct effects of the mediator on the neurons and the therapeutic effect of therapies cannot be observed. For example, nerve growth factor (NGF), interleukin-6 (IL-6), endothelin-1 (ET-1), tumor necrosis factor alpha (TNF-a), bradykinin (BK), adenosine triphosphate (ATP), H+, and trypsin each affect receptors expressed in dorsal root ganglion (DRG) neurons and, accordingly, can induce measurable responses in DRG neuron models.

Another criterion may be that the mediator causes local pain in animal models. For example, local pain can be measured through injection of rodents with the mediator (e.g., with measurements of post-injection licking, shaking, or chewing behavior, or through observed withdrawal threshold).

Similarly, known pharmacological blocks of the mediator-associated receptor can be tested in animal models to attempt to alleviate pain associated with the mediator. In certain embodiments, successful pain alleviation with known pharmacological blocks may be a criterion for selection of a mediator for inclusion in a composition of the invention. Additionally, given the availability of, or ability to create, a mouse knockout that has been genetically modified to not express the receptor associated with a particular pain mediator, that knockout mouse may be tested via local injection with the candidate mediator. In certain embodiments, a lack of pain symptoms in injected knockout mice may be a factor in selecting a mediator for inclusion in a pain mediator composition.

Once the general composition of a pain mediator composition has been determined, specific concentrations of each component mediator may be determined. In certain embodiments, concentration may correspond to the measured concentration levels of the mediator in tumor-associated samples. Concentrations may also be determined experimentally using assays of the invention. Each component must be present in the pain mediator composition at a concentration sufficient to elicit a measurable response in cultured neurons in order to function in therapy screening. Accordingly, neurons (preferably expressing optogenetic proteins), may be exposed to varying concentrations of a mediator and observed for measurable response. The neurons may be exposed to varying concentrations of a mediator alone or may be exposed to a mediator mixture in which the concentration of one or more of mediators is varied. A concentration of each mediator may then be selected that corresponds to at least the minimum concentration that induces an observable response in cultured neurons.

Screening models of the invention may include dorsal root ganglion (DRG) neurons or sensory neurons exposed to a pain mediator composition of the invention. For example, a screening model for cancer pain therapeutics may use a cancer pain mediator composition that is a general, generic pain mediator composition or may be selected to model the cancer microenvironment of specific cancer types. The neurons used may express optogenetic proteins that allow neural activity to be detected optically such that the effects of the cancer pain mediators and potential therapies can be monitored optically. Exemplary systems and methods of screening pain-treating compounds using optogenetically modified neurons are disclosed in U.S. patent publication number 2015/0301028, incorporated herein by reference.

Cancer pain mediator compositions (i.e., “cancer pain soup” formulations), according to the invention, may include any number of mediators meeting some combination of the criteria discussed above. For example, a pain mediator composition may include one or more of nerve growth factor (NGF), endothelin-1 (ET-1), tumor necrosis factor alpha (TNF-a), bradykinin (BK), interleukin-6 (IL-6), adenosine triphosphate (ATP), H+, and trypsin. Pain mediator compositions may include two or more of NGF, ET-1, TNF-a, BK, IL-6, ATP, H+, and trypsin. In certain embodiments, a pain mediator composition may include three or more of NGF, ET-1, TNF-a, BK, IL-6, ATP, H+, and trypsin. In some embodiments, a pain mediator composition of the invention may include four or more of NGF, ET-1, IL-6, TNF-a, BK, ATP, H+, and trypsin. Pain mediator compositions may include five, six, or seven of, or each of, NGF, ET-1, IL-6, TNF-a, BK, ATP, H+, and trypsin. In certain embodiments, the above pain mediator compositions may additionally comprise one or more of formaldehyde, Substance P (SP), and brain-derived neurotrophic factor (BDNF).

Concentrations of various mediators within the composition may be as follows. NGF may be present in a composition of the invention at a concentration of about 60 μg/mL as measured experimentally in supernatant of human oral cancer cells. ET-1 may be present in a composition of the invention at a concentration of about 30 nM-about 10 μM. TNF-a may be present in a composition of the invention at a concentration of about 0.4 ng/mL as measured experimentally in tumor supernatant. IL-6 may be present in a composition of the invention at a concentration of about 4 ng/mL as measured experimentally in tumor supernatant. BK may be present in a composition of the invention at a concentration of about 5 μM. ATP may be present in a composition of the invention at a concentration of about 100 μM. H+ may be present in a composition of the invention at to bring the composition pH to about 6.5. The pH of the composition may be about 6.1 at 37 degrees Celsius and about 5.4 at 22 degrees Celsius. Trypsin may be present in a composition of the invention at a concentration of about 1 μg/mL or about 42 nM.

### Example 7: Pharmacology and Multidimensional Analysis

FIGS. 26-29 shows results from an assay of pharmacology and multidimensional analysis. E18 rat hippocampal neurons were cultured 14 days.

FIG. 26 shows Radar plots showing the drug-induced change in neuronal spiking behavior along many dimensions, tabulated below the plots. For each parameter, the vehicle is normalized to 1 (labeled vehicle), and the drug (labeled drug) shows the fold change caused by drug application. FIGS. 27-29 outline one strategy for combining the multidimensional readout into a single number for, e.g., drug screening. The recordings during the stimulus protocol are partitioned into coarse time bins, 48 bins for this protocol (see blue stim below FIG. 28). In each time bin, the average spike rate, spike height, spike width, and spike after hyperpolarization (AHP) depth are calculated for each well. Each parameter is normalized to the time and population average, and the parameters are concatenated together to yield a single 1×192 (4*48) vector capturing the behavior of each well.

FIG. 27 shows measured action potentials for 6 wells treated with XE-991 and 6 vehicle control wells. Locations where “vehicle” and “drug” traces separate highlight parameters and stimuli where drug effects are most obvious. To convert traces into a single number, a weight vector is calculated that selects for regions with a large drug-induced effect and selects against parameters that are noisy: wt=(<drug>−<ctrl>)/(var(drug)+var(ctrl)).

FIG. 28 shows the trace for each well after subtraction of the average control trace and multiplication with the weight vector. It is visually obvious where to discriminate between drug and control (the drug traces hover around the x-axis, the vehicle traces are the high spikes).

FIG. 29 shows, for each well, the drug score is calculated by summing over the trace in FIG. 28. The vehicle wells are the same for each drug, but the weight vectors are different. For all drugs, the difference can be robustly detected. Z′=1−3*(std(drug)+std(ctrl))/abs(<drug>−<ctrl>) is a measure of statistical effect size, and is large enough to robustly detect each of these drug effects. In all cases, the vehicle plot points are in the top half of the panels and the drug plot points are in the bottom half.

### Example 8: Validation of High-Throughput Screening

FIGS. 30-34 show high-throughput validation for screening. E18 rat hippocampal neurons cultured for 14 days.

FIG. 30 shows high signal-to-noise ratio fluorescent voltage recordings of neurons on the 96-well plate (in a microscopy instrument of the disclosure) in vehicle control wells or in wells with 1 μM ML-213, a Kv 7.x agonist that hyperpolarizes cells and reduces firing. The blue light stimulus is shown below.

FIG. 31 is a raster plot showing spikes from columns of wells. ML213 dramatically reduces firing rate in all wells tested.

FIG. 32 shows the average firing rate during the ramp for each well. Vehicle wells (even columns, green) and ML213 wells (odd columns, red) are easily distinguished. Even numbered columns are all green; odd numbered columns are all read.

FIG. 33 gives a heat map showing the number of spikes during the ramp for each well. The columns line up with the columns of FIG. 2 and those column headers apply to FIG. 33.

FIG. 34 shows the average number of spikes per cell for the ramp portion of the protocol for each. The calculated Z′ of 0.31 is more than good enough to execute a phenotypic screen.

### Example 9: High-Throughput Measurements in Different Cell Types

FIGS. 35-37 are from high-throughput measurements in different cell types. Fluorescent voltage recordings showing excellent signal to noise and spike detection for (DRG) neurons and for NGN2 neurons.

FIG. 35 shows measurements from rat dorsal root ganglion (DRG) neurons.

FIG. 36 is a raster plot showing the resulting spike data for rat DRG neurons exposed to a pain mediator composition comprising 5 inflammatory mediators. The pain mediator composition leads to a dramatic increase in spike firing rate in all wells tested.

FIG. 37 shows measurements from human iPSC-derived NGN2 neurons. The assay fidelity for screening is calculated with multi-parameter reduction to a single value.

FIG. 38 gives the single values resulting from rat DRG neurons (left panel) and FIG. 38 human NGN2 neurons (right panel).

### Example 10: One Embodiment of an Osteoarthritis Pain Mediator Composition

FIG. 25 gives a recipe for an osteoarthritis pain mediator composition according to one embodiment.

### Example 11: Use of Pain Mediator Composition

High-throughput, all-optical electrophysiological recordings are applied to sensory neurons derived from rodent tissue in the context of novel in vitro models of pain incident to cancer, inflammation, neuropathy, and other diseases. The electrical properties of neurons may be recorded with Optopatch, which uses genetically encoded proteins for all-optical studies of the cell transmembrane potential (FIG. 8; described in more detail below). Blue light causes the channelrhodopsin CheRiff to open, triggering action potentials, while red light excites the fluorescent voltage sensor QuasAr to record the electrical activity. Optopatch measurements maintain the rich information content of manual patch clamp, but with vastly higher throughput. This platform enables fast, efficient and consistent recordings of intrinsic neuronal excitability, synaptic transmission, and network behavior.

Methods of the disclosure provide for the generation of models for pain, including those specific to inflammation, neuropathy and cancer.

Cultured DRG neurons show robust firing phenotypes, including an acute response to capsaicin and hyper-excitability in response to an inflammatory pain mediator composition (a mixture of histamine, prostaglandin, serotonin, ATP, and bradykinin, soluble mediators of inflammatory pain; FIG. 13 for example in rodent DRG). This approach serves as a basis for a phenotypic screen for compounds that ameliorate differences in behavior between “inflamed” and healthy cells. It could also be used as a secondary assay for a target-based screen to confirm that hit compounds are effective in a neuro-physiological setting. The throughput is sufficient to drive chemical optimization.

An analogous mixture of the tumor- and immune cell-released mediators of chronic cancer pain is used as a cancer pain mediator composition. When added to media containing cultured DRG neurons, the cancer pain mediator composition may be used to establish a cancer pain excitability phenotype and synaptic assays for sensory neurons to screen for compounds that ameliorate the pain phenotype.

Any suitable electrophysiological and/or fluorescent methods that can be applied to the study of cancer pain, inflammatory pain, neuropathic pain and other disease conditions that induce pain in vitro may be used with methods of the disclosure. For example, suitable technologies may include the automated patch clamp technologies provided by Nanion, Sophion, or MDC/Ionworks. Suitable technologies may include a multi-electrode array, e.g., as provided by Axion Biosystems. Suitable technology may include calcium imaging as provided by Vala Biosciences. Preferred embodiments use optogenetic assays such as Optopatch with CheRiff and QuasAr. Preferred optogenetic embodiments offer single-cell precision, large numbers of cells recorded and beneficial fluorescent reporters used.

A suitable microscope shown in FIG. 6 and FIG. 7, provides simultaneous Optopatch recordings from as many as 100 neurons with 1 millisecond temporal resolution and sub-cellular spatial resolution in a 4 mm×0.5 mm field of view (FOV), and may be used with multiple types of primary rodent and human induced pluripotent stem (hiPS) cell-derived neurons, including sensory neurons (see FIG. 9 below for demonstration of differentiation and functional recordings with Optopatch). Though current recordings are performed in single- or 8-well format it may be preferable to use a 96/384 well plate version of the microscope 600, which features automated drug delivery and the ability to perform these assays in high-throughput format. The microscope 600 uses a custom prism to couple a high-power laser into the sample to eliminate background fluorescence introduced by the objective lens, and uses a custom imaging path to record a large FOV at high speed while maintaining high light collection efficiency (FIG. 7). A key feature of the microscope 600 microscope is a digital micromirror device 653 that contains around 800,000 individually addressable micromirrors, each of which directs optical stimulation to a distinct spot on the sample. This allows optogenetic stimulation in arbitrary patterns of space and time. In this way, one can stimulate one or more pre-synaptic neurons and record from post-synaptic partners.

After a movie is recorded using the microscope 600 (typically 15 seconds of recording at 1000 frames/sec), it is transferred to a computer system (e.g., Amazon Web Services (AWS)) for multiply-redundant data storage. Once an entire dataset is uploaded to the computer system, hundreds of processors are simultaneously spooled up, each of which analyzes a single movie. An arbitrarily large dataset can be analyzed in 1 hour.

FIG. 13 shows a representative experiment in which the activity of 174 mouse DRG neurons in culture from two imaging wells was recorded prior to intervention (top trace for each cell), after application of a pain mediator composition (middle trace) and subsequent application of 1 μM TTX (bottom trace). Custom-built analysis software automatically parses the millions of imaging frames collected, creates voltage traces for each recorded neuron and identifies each action potential fired. Dataset plots are automatically generated, of which a small selection are shown on the bottom half of FIG. 13. As FIG. 10 shows, application of the pain mediator composition increases stimulus-induced neuronal firing approximately 3-fold (lower-left panel) but does not alter the spike width or after-hyperpolarization depth. TTX, which blocks Nav1.7 but not Nav1.8/1.9 channels in these neurons, blocks the effect of the pain mediator composition, increases spike width and decreases after-hyperpolarization depth.

The changes in cellular excitability produced by the pain mediator composition are used as the basis for a phenotypic screen. FIG. 39 shows the assay reliability. The change in the average number of spikes per cell upon addition of the pain mediator composition (“x”, across the top) or vehicle (“+”, across the bottom), showing clear separation in single-well measurements.

FIG. 39 shows how the number of spikes during a staircase-stimulus recording (as shown in FIG. 13) changes upon addition of the pain mediator composition. There is no overlap between vehicle and pain mediator composition data points when averaging over a single well, demonstrating a robust screening phenotype.

While it is may be believed that cancer pain is caused by physical pressure from the tumor, evidence points to chemical or biological mediators of cancer pain. FIG. 4 shows the complex milieu of pain signal factors present in the tumor microenvironment. Signaling can directly mediate excitability via ionotropic receptors such as P2X3, TRPV1, or ASIC, or can cause longer-term changes in excitability or synaptic release via signaling cascades triggered by metabotropic receptors such as TNFR or TrkA. A pain mediator composition may be identified to reproduce the cancer microenvironment in vitro to enable rapid screening of agents that mitigate cancer pain. The cellular pain model is combined with assays for DRG neuron excitability and synaptic transmission to build phenotypic pain assays. Those assays serve drug screening efforts to identify novel molecules that mitigate the phenotype. We hypothesize that since multiple mediators are involved in generating cancer pain, a phenotypic assay, rather than a target focused approach, provides the best opportunity to identify agents that minimize a complex cellular phenotype.

One aspect of the disclosure provides a synaptic assay for sensory neurons. Using methods of the disclosure, the microscope 600 measures changes in neuronal intrinsic excitability with high precision. This is a promising mode of pharmacological intervention: a compound that reduces sensory neuron firing rate in response to a noxious stimulus are likely analgesics. However, synaptic transmission between primary afferent sensory cells and neurons in the dorsal horn of the spinal cord provides an additional promising point for pharmacological modulation.

A first embodiment of the one aspect provides a synaptic vesicle release assay. Protein-based pH sensors targeted to the inner leaflet of synaptic vesicles are used to track vesicle release. For example, the pHlorans are pH-sensitive mOrange variants with fine-tuned pKas. In the pH 5.5 environment of the vesicle, the fluorescent sensor is dim. Upon release of vesicle contents into the synaptic cleft, the pH increases to 7.4 and the sensor brightens several-fold. Following development of the green pHluorin sensor, the red pH sensor pHuji may be targeted to synaptic vesicles for use in concert with blue-light activated CheRiff. The pHuji sensor is described in Shen, 2014, J Cell Biol 207(3):419, incorporated by reference.

FIG. 40 shows synaptic assays. In panel A, in a vesicle cycling assay, the DRG neurons express CheRiff for stimulation and the synaptically-targeted red pH sensor pHoran for readout. In panel B, pHoran traces from 8 wells during tonic electrical stimulus. In panel C, for a synaptic transmission assay, DRG cells express CheRiff and post-synaptic dorsal horn neurons express QuasAr for readout. In panel D, EPSP recordings with no drug (left), 20 μM cyclothiazide, a positive AMPA modulator (middle), and 100 μM GYKI 53655, a negative AMPA modulator (right). The cells are E18 rat hippocampal cultured neurons.

Blue light is used to trigger neuronal activity, and orange light is used to record vesicle release and recycling via signal amplitude and recovery time (FIG. 40, panel A). The assay is pharmacologically validated using channel blockers to block Cav2.1, 2.2, & 2.3 and prevent vesicle release, and the phorbol ester PMA to upregulate vesicle release. DRG vesicle release is an important phenotypic endpoint, so the assay is adapted from rat hippocampal neurons (FIG. 40, panel B; electrical stimulation) to DRG neurons. A pre-synaptically targeted red calcium sensor jRGECO1a14 may be used as an alternative readout of presynaptic activity.

In another embodiment, the disclosure involves synaptic transmission via excitatory post-synaptic potentials (EPSPs). As an alternative and complimentary method to measure DRG synaptic signaling, EPSPs are measured in dorsal horn neurons. The actuator CheRiff is expressed exclusively in the DRG neurons, and the voltage sensor QuasAr is expressed exclusively in the dorsal horn neurons (FIG. 40, panel C). Full-field blue light stimulation evokes action potentials in the DRG neurons, and transmitted EPSPs are detected in the dorsal horn neurons, allowing measurement of a key event in pain signaling. EPSP amplitude, rise time, and decay times are recorded for each dorsal horn neuron. The assay is pharmacologically validated with AMPA and NMDA channel blockers APV and NBQX and positive allosteric modulators LY404187 and pregnenolone. This assay is adapted from a synaptic assay developed in cultured E18 rat neurons, where both pre- and post-synaptic cells are from the hippocampus (FIG. 40, panel D).

Implementation may involve a staged dissection protocol in combination with Cre-recombinase-dependent fluorescent constructs. DRG neurons are plated and transduced immediately with two lentiviruses, one encoding CheRiff and the other encoding Cre. Those viruses transduce neurons with very high efficiency, so >95% of DRG neurons express both CheRiff and Cre. During dissection, the spinal cord is reserved and stored in Hibernate A medium (Brain Bits #HA) at 4° C. After two days, the dorsal region of the spinal cord is removed to dissociate neurons, and plate on top of the cultured DRG neurons. After cells are plated, they are transduced with a Cre-OFF-QuasAr lentivirus. The sensor is flanked by loxP sites, excised, and turned off in cells expressing Cre. In the dorsal horn neurons, where no Cre is present, the QuasAr is expressed normally. In the DRG neurons, Cre recombinase activity excises the QuasAr construct and the sensor is not expressed. Thus, methods of the disclosure exclusively stimulate DRG neurons and exclusively measure from dorsal horn neurons. The presence of CheRiff-triggered PSP's may be validated with manual patch clamp and synaptic blockers. As an alternative assay, use post-synaptically targeted red calcium sensor jRGECO1a.

Another aspect provides in vitro models for cancer pain. A cancer pain mediator composition (i.e., a “cancer pain soup”), a mixture of signaling molecules found in the tumor microenvironment typified by those shown in FIG. 4, is provided. When DRG cells are incubated chronically with a cancer pain mediator composition, their excitability and synaptic strength is modulated, analogous to the effect of an inflammatory pain mediator composition. Here, the disclosure provides a method that includes selecting pain mediators and combining the mediators to create a pain mediator composition. There are a number or criteria for including a pain mediator as an ingredient in the composition: First, in humans, the mediator must be detected at increased levels in or near tumors. The more tumor types it has been associated with, the better. Second, the associated receptor must be expressed in DRG neurons. Third, when injected in rodents, it must cause local pain. Fourth, after injection of the pain mediator, a pharmacological blocker of the associated receptor must alleviate pain in the injected rodent. For a fifth criterion, ideally, a receptor agonist will enhance pain in the rodent. Sixth, ideally, injection will cause less pain in a mouse KO of the receptor. And lastly (e.g., 7), ideally, the measured concentration of the mediator in the tumor microenvironment will correlate with clinically reported pain levels. Data on all 7 criteria are not available for most pain mediators, but criteria 1-4 are considered essential. Initial concentrations are set by measurements in the tumor microenvironment, where data are available, and at levels that induce pain in vivo otherwise. FIG. 41 shows Table 2, which shows the current formulation, and FIG. 42 shows preliminary data suggesting a hyperexcitability effect.

FIG. 42 shows a cancer pain mediator composition phenotype in DRG neurons. The figure gives a preliminary raster data plot showing increased spiking from DRG neurons that were treated with a cancer pain mediator composition (formulation from Table 2).

Technical methods may be used to optimize a formulation of a pain mediator composition. Starting with a best estimate for the initial formulation, methods may include experimental optimization. The microscope 600 allows for measuring up to 96 formulations per plate using either the set of excitability readouts (FIG. 13) or synaptic readouts. Optimization may include generating concentration-response curves (CRC) for each ingredient in isolation (e.g., FIGS. 17-20), as well as a CRC for each ingredient in the presence of the other seven, all at two incubation time points (30 min and 24 hours) and in duplicate (using the plating of, e.g., FIG. 12). Ingredients that have no effect within the canonical physiological concentration range are removed from consideration. Other concentrations are adjusted to their estimated IC50, so all components are relevant and the complex signaling interactions are captured. Iterate 3 times, as the IC50 of each pain mediator component likely depends on the concentration of the other components. Measure the IC50 values in the formulation using both excitability and synaptic assays, and preferably set the final concentration to the logarithmic mean of the two values. After cellular exposure to the pain mediator composition, induced changes in rheobase excitability and action potential waveforms may be determined in current clamp experiments, and sodium, calcium and potassium channel currents may be characterized in manual voltage clamp experiments using established protocols.

Another embodiment provides a chemotherapeutic pain model. As an alternative to the cancer pain mediator composition, and as an important cause of pain in patients, the disclosure provides a cellular model of cancer chemotherapeutic pain using paclitaxel and cisplatin. Paclitaxel causes dose-limiting sensory neuropathies and neuropathic pain. Cisplatin causes hyperalgesia (increased sensitivity to pain), and has been shown to cause neuronal hyperexcitability in vivo with extracellular voltage recordings. Testing may be done with the excitability and synaptic assays at different concentrations (clinical dose (CD), CD/5, & CD*5) and at time points following literature precedent.

Embodiments provide validation for cancer pain mediator compositions in human differentiated sensory neurons. A finalized formulation of a cancer pain mediator composition is validated as are chemotherapeutics in cultured human stem cell-derived neurons with sensory neuron-like properties. An induced pluripotent stem (iPS) cell line is differentiated into sensory neurons using an established protocol (FIG. 43).

FIG. 43 shows iPS cell-derived sensory neurons. In panels A and B, immune-cytochemistry (ICC) assays show expression of the pan-neuronal marker MAP2 and the sensory neuron transcription factor ISLET1, as well as the PNS intermediate filament Peripherin (PRPH). 95% of cells are MAP2+, and 75% of cells are ISLET1+/PRPH+. In panel C, a raster plot (one row per cell, one dot per action potential) shows spiking in these neurons in response to the blue stimulus. Cells fire robustly before toxin addition (“Pre”), and almost all spiking is blocked after addition of 1 μM tetrodotoxin (“Post”).

This approach relies on small molecule inhibition of dual SMAD signaling to produce neural progenitors that are then patterned into sensory neuron-like cells by combined inhibition of FGF and NOTCH and activation of WNT signaling pathways. Immunocytochemistry (ICC) and functional assays show efficient production of cells expressing sensory neuron markers; the cells display robust firing and sensitivity to TTX (FIG. 43). Methods test whether the cancer pain mediator composition and chemotherapeutics modulate excitability similarly in hiPS cell-derived sensory neurons and in primary mouse DRG neurons.

Aspects provide for validation of a cancer pain mediator composition in vivo. To confirm that the composition has in vivo relevance, it may be tested in a mouse cancer pain model and compared against vehicle (negative control), complete Freund's adjuvant (CFA) (positive control), and the supernatant of the squamous cell carcinoma (SCC) cell line (positive control). SCC is a particularly painful type of oral cancer that has been used extensively in cancer pain research. The testing may be performed by an independent laboratory using classical tests for thermal hyperalgesia and mechanical allodynia (von Frey filament tests). Those experiments test whether the mouse's hind paw injected with the insult becomes more sensitive to touch or temperature than the paired paw injected with vehicle. A suitable test may use 10 mice/condition (5 male mice and 5 female mice to consider sex as a biological variable) to achieve robust, statistically significant results. As an alternative mouse pain model, a test may use the Dolognawmeter (time to gnaw through a dowel after tongue injection), previously developed for cancer pain experiments. The gnaw time is compared between mice whose tongues are injected with vehicle compared to insult. If the cancer pain mediator composition shows no effect at the design amount or at 3× design amount, SCC supernatant is used (produced in large batches, aliquoted, and frozen), already demonstrated to cause pain in mice, for an in vitro cancer pain model.

Aspects of the disclosure provide methods to probe potential pain targets using established pharmacology. Methods may be used to test a functionally broad set of compounds to identify reversals of the cancer pain phenotype in vitro in rodent primary neurons. A phenotype may be considered to be the difference in excitability or synaptic activity between wells with and without a cancer pain mediator composition or chemotherapeutic treatment. Methods may include initially testing at 3 concentrations of each compound in duplicate (IC50, 3×IC50, and 10×IC50). Compounds showing effects may be confirmed with an 8-point concentration-response. Confirmed hits may be counter-screened for effects on hippocampal neurons; drugs that ameliorate the cancer pain phenotype without compromising the central nervous system highlight promising drug targets. FIG. 44 gives Table 3, which shows probe compounds expected to modulate pain signaling, excitability, or synaptic transmission.

Methods and techniques of the disclosure provide tools for screening excitability and synaptic phenotypes, providing a path to an in vitro cancer pain model with throughput to support drug discovery efforts. An assay using a cancer pain mediator composition (i.e., a cancer pain soup assay) may be used to conduct a large phenotypic screen for novel cancer pain therapeutics.

### Example 12: Data from Osteoarthritis Pain Mediator Composition

FIG. 45 gives data from an initial formulation of the osteoarthritis pain mediator composition. The figure shows 48-hour treatment with three different concentrations. The larger number of ticks at the higher concentration, each representing an action potential, indicates hyperexcitability of the neurons. Those data show that the pain mediator composition has a measurable and demonstrable effect on neural activity and neural phenotype, and that methods, instruments, reagents, and techniques of the disclosure are useful and effective for measuring an effect of a pain mediator composition on neural activity and neural phenotype.

### Example 13: DRG/Dorsal Horn Synaptic Assays

In certain embodiments, methods and compositions of the disclosure are used with measurement of the strength of synapses from the DRG neurons into the dorsal horn. Chronic neuropathic pain is thought to result from long-term changes at the synapse of DRG into the dorsal horn and some therapeutics, such as opioids, act in large part at that synapse.

Techniques of Example 11 above are applied here. Methods include providing a sample in which dorsal root ganglia are in synaptic communication with a dorsal horn or neurons or grey matter characteristic of the dorsal horn.

The dorsal horn is one of the three grey columns of the spinal cord and receives sensory information from receptors of the skin, bones, and joints through sensory neurons whose cell bodies lie in the dorsal root ganglion (DRG). Neurons in the spinal dorsal horn process sensory information, which is then transmitted to several brain regions, including those responsible for pain perception. Neurons of the DRG may lie within the DRG but outside of the axonal path at the end of a “t-junction” of the axon, with axons that are split peripherally and centrally. The T-junction of the DRG neuron can either act as a barrier to the propagation of action potentials (APs) to the dorsal horn, as a filter of APs, or helps to propagate APs to the DH.

Methods and techniques of the disclosure may be used to make measurements at a synapse from the DRG neurons into the dorsal horn, e.g., in vivo or in an in vitro model.

FIG. 40 in panel C illustrates a neural plating for measuring a synapse between a pre-synaptic DRG and a post-synaptic neuron of the dorsal horn. Panel D shows measurements that may be made. For neuropathic pain in particular, as well as for other pain models, measuring the strength of synapses from the DRG neurons into the dorsal horn may provide insights into, for example, the potential usefulness of a compounds as an analgesic.

Any suitable metric may be used including, for example, neural firing. It may be beneficial to measure any suitable aspect of an action potential readout such as, for example, changes in the number of action potential, changes in the minimum stimulus required to evoke an action potential (the rheobase), a change in spike shape, or a change in synaptic properties. FIG. 11 illustrates such measurements.

Any suitable electrophysiological and/or fluorescent methods may be used for measuring strength of synapses from the DRG neurons into the dorsal horn. For example, suitable technologies may include the automated patch clamp technologies provided by Nanion, Sophion, or MDC/Ionworks. Suitable technologies may include a multi-electrode array, e.g., as provided by Axion Biosystems. Suitable technology may include calcium imaging as provided by Vala Biosciences. Preferred embodiments use optogenetic assays such as Optopatch with CheRiff and QuasAr. A suitable microscope shown in FIG. 6 and FIG. 7, provides simultaneous Optopatch recordings from as many as 100 neurons with 1 millisecond temporal resolution and sub-cellular spatial resolution in a 4 mm×0.5 mm field of view (FOV), and may be used in measuring neural activity at synapses from the DRG neurons into the dorsal horn.

Any composition of the disclosure may be used to measure an effect of a drug on a strength of synapses from the DRG neurons into the dorsal horn when associated with a painful condition. For example, FIG. 25 gives a recipe for an osteoarthritis pain mediator composition according to one embodiment.

Thus, it may beneficial to evaluate an effect of a compound on neural activity across synapses from the DRG neurons into the dorsal horn. This may include providing a sample comprising one or more DRG neurons in synaptic communication with a post-synaptic neuron of the dorsal horn, as shown in FIG. 40. The sample may be in media comprising a plurality of pain mediators associated with a condition such as osteoarthritis (e.g., a composition according to the recipe of FIG. 25). An effect of the compound in the presence of the pain mediators on neural activity in the sample is measured.

For example, with a radar plot such as that shown in FIG. 11, one may measure a change in the minimum stimulus required to evoke an action potential (the rheobase) or a change in spike shape. When a compound mitigates an effect of the mediators on neural activity at the DRG synapse, that compound may be identified as a candidate analgesic.

Development of a compound that mitigates an effect of a pain composition on activity across the DRG/dorsal horn synapse may provide treatments with significant pain relief for people suffering from pain or inflammatory conditions.

1-14. (canceled)

15. A composition comprising:
a plurality of pain mediators that are present in or near a target tissue associated with a pain response.

16. The composition of claim 15, in which the pain mediators are present in relative concentrations that approximate the relative concentrations of such mediators in the target tissue.

17. The composition of claim 16, in which the target tissue is a tumor.

18. The composition of claim 17, in which the mediators include four or more nerve growth factor (NGF), endothelin 1 (ET-1), tumor necrosis factor alpha, interleukin 6, adenine triphosphate, bradykinin, acidic pH, and a protease.

19. The composition of claim 16, in which the target tissue is nerve tissue.

20. The composition of claim 19, in which the mediators include four or more tumor necrosis factor alpha, nerve growth factor (NGF), interleukin (IL)-1beta, IL-6, IL-17, acidic pH, PGE2, and CCL2.

21. The composition of claim 16, in which the target tissue is affected by inflammation.

22. The composition of claim 21, wherein the target tissue is osteoarthritic tissue.

23. The composition of claim 22, in which the mediators include four or more of tumor necrosis factor alpha, nerve growth factor (NGF), interleukin (IL)-1beta, IL-6, IL-17, acidic pH, PGE2, and CCL2.

24. The composition of claim 15, wherein the mediators include at least one interleukin, at least one growth factor and at least one other mediator.

25. The composition of claim 15, wherein the mediators satisfy one or more of the following: (a) the mediator triggers an action potential in a neuron; (b) the mediator increases sensitivity to a pain response in a neuron; (c) the mediator triggers a pain response in an animal model; (d) when the mediator is administered to an animal for which the associated receptor gene is knocked out, there is no pain response.

26. The composition of claim 15, wherein administration of the composition to an animal causes a pain response in the animal.

27. The composition of claim 15, wherein the composition includes living neurons.

28. The composition of claim 27, wherein the neurons and the plurality of pain mediators are provided in media ex vivo.

29. The composition of claim 28, wherein at least one of the neurons includes an optical reporter of neural activity.

30. The composition of claim 29, wherein the optical reporter includes a microbial rhodopsin protein.

31. A method comprising:
inducing a pain response in a neuron by contacting the neuron with a pain mediator composition which includes pain mediators that are present in or near a target tissue associated with a pain response.

32. The method of claim 31, wherein the pain mediator composition includes a plurality of soluble proteins.

33. The method of claim 31, wherein the method is performed in vivo by administering the pain mediator composition to an animal.

34. The method of claim 33, wherein the pain response includes pain-associated behavior by the animal.

35-84. (canceled)
